(19) United States
(12) Patent Application Publication
(10) Pub. No.: US 2026/0317764 A1
(43) Pub. Date: Oct. 15, 2026

(54) DESIGN AND METHOD OF GENERATING SCARLESS CIRCULAR RNA

(71) Applicant: Sekkei Bio Private Limited, Taramani, Chennai (IN)

(72) Inventors: Monalisa CHATTERJI, Bengaluru (IN); Anirudh RANGANATHAN, Chennai (IN); Srivatsa DWARAKANATH, Bangalore (IN); Anand Prakash KHEDKAR, Bangalore (IN); Asma Nadhira Haseen MEERA MOHAIDEEN, Madurai (IN)

(21) Appl. No.: 19/483,214

(22) PCT Filed: May 9, 2024
(86) PCT No.: PCT/IB2024/000261

§ 371 Date: Nov. 10, 2025

(30) Foreign Application Priority Data
May 12, 2025 (IN) 202341033449

(51) Int. Cl. C12N 15/113

(52) U.S. Cl. CPC C12N 15/113; C12N 2310/1241; C12N 2310/532; C12N 2330/50

(57) ABSTRACT

A method of making circularization of an RNA of interest without a use of permuted intron-exon (PIE) includes: providing a DNA (deoxyribonucleic acid) construct, wherein the DNA construct includes a Group I intron-based ribozyme, a sequence of interest, and a target sequence; causing an RNA to be transcribed from the DNA construct, the RNA includes a segment S1 corresponding to the ribozyme, a segment S2 corresponding to the target sequence, and a segment S3 corresponding to the sequence of interest; and effecting a base-pairing between the segment S1 and the segment S2, wherein an occurrence of the base-pairing induces circularization of the segment S3.

### CROSS-REFERENCE TO RELATED APPLICATION(S)

This application claims priority from Indian Provisional Application Number IN202341033449, filed on May 12, 2023, entire contents of which are incorporated herein by reference in their entireties.

### TECHNICAL FIELD

The present disclosure relates to the field of molecular biology, and in particular, relates to methods for construction of scarless circular RNA generating constructs using Tetrahymena or Twort ribozyme, or one or more of their respective variants.

### SEQUENCE LISTING SUBMITTED ELECTRONICALLY

Accompanying the present disclosure is a Sequence Listing submitted electronically in XML format (file name 1134-002-PCT.xml, file size 38 kilobytes, and date of creation May 2, 2024). The information in the electronic Sequence Listing is incorporated herein by reference in its entirety. If and when a discrepancy exists between a sequence expressly stated in the following text of the present disclosure and a sequence stated in the Sequence Listing in XML format, the sequence expressly stated herein in the present disclosure is correct.

### BACKGROUND

With recent approval of mRNA vaccine against SARS-COV-2 during the corona virus pandemic (N. Engl. J. Med. 2021, 384, 403-416), RNA vaccines and therapeutics are in demand. Ribonucleic acid (RNA) is a polymeric nucleic acid molecule important for various biological functions such as coding, decoding, regulation and expression of genes. RNA is usually a single stranded molecule. In the late 1970s, Hsu and Coca-Prados (Nature 1979, 280, 5720, 339-340) have shown that there is a single stranded covalently closed, circular form of RNA expressed throughout the animal and plant kingdom.

Circular RNA (circRNA) is a type of single stranded RNA where the 3′ and 5′ ends have been joined together. Because circular RNA does not have 5′ or 3′ ends it is resistant to digestion by exonuclease. Circular RNAs have been discovered in many different eukaryotic groups, from fungi to fish, birds to insects, and plants to protists. Of the more than 100,000 human circular RNAs that been discovered to date, functionality has been attributed to only a handful.

Circular RNA may be generated in vitro using an enzymatic method. Enzyme based ligation methods employ ATP-dependent T4 DNA ligase or T4 RNA ligases. The ligases are used to catalyze the reaction for joining of RNA termini, namely the 5′ phosphate and 3′ OH groups through three nucleotidyl transfer steps (Structure 2004, 12, 327-339).

Circular RNA may be generated in vitro using a chemical method. In the chemical method, representative reactions of phosphodiester bonds formation in the presence of phosphate activating agents, such as cyanogen bromide (BrCN) or water-soluble ethyl-3-(3′-dimethylaminopropyl)-carbodiimide (EDC) are summarized by Muller and group (Nucleic Acids Res. 2015, 43, 2454-2465). Chemical ligation may introduce biosafety concerns and therefore is met with limitations.

Circular RNA may also be generated in vitro using a ribozyme enabled method, where group I intron self-splicing system such as permuted intron-exon (PIE) method is a ribozyme-enabled method. In the PIE method, the RNA segment to be circularized is flanked by two halves (in same or different sequence size) of the split group I intron. Group II introns may also be used for circRNA synthesis which involves inverse splicing reaction. Hairpin ribozymes, which may catalyse self-cleavage and ligation reactions can also be used to produce circular RNAs via the rolling circle replication reaction (RNA Biology 2017, 14, 8, 1018-1027).

Group I introns are intronic sequences that have the ability to undergo self-splicing, for example, catalyze their own excision from flanking exon sequences. Self-splicing of the intron involves a two-step trans-esterification pathway which involves RNA cleavage followed by ligation. A non-encoded guanosine cofactor initiates the splicing reaction by carrying out a nucleophilic attack and creating a nick at the 5′ end of the intron region. The attacking guanosine cofactor is covalently attached to the released intron at the 5′ end. Next, the free 3′ end of the released upstream exon attacks the 3′ end of the intron resulting in the release of the intron and ligation of the exon sequences, as illustratively shown in FIG. 1A. FIG. 1A is a schematic diagram, showing a mechanism of cis-splicing activity of the ribozyme, where the reaction begins with the nucleophilic attack by the hydroxyl group of guanosine on the 5′ splice site, and where the free 3′ end of the released exon attacks the 3′ end of the ribozyme resulting in its ligation to the 3′ exon and the release of the ribozyme. The borders between exons and most group I intron are defined at the RNA structural level. At the sequence level, the last nucleotide in the upstream exon is often a U (uracil), and the last nucleotide of the intron is often a G (guanine).

Group I introns are often found to be cis-acting. However, by removing the 5′ exon and modifying the 5′ terminus, group I introns-based ribozymes with trans-splicing ability may be generated, as illustratively shown in FIG. 1B. FIG. 1B is a schematic diagram, showing a mechanism of trans-splicing activity of the ribozyme, where although similar in mechanism to cis-acting Group I introns, trans-splicing ribozymes carry modifications at their 5′ end. Such trans-acting group I intron ribozymes may be used to cleave a target substrate RNA, insert or remove one or several nucleotides, to replace the 3′ portion of the substrate RNA with the 3′ exon of the ribozyme, or to replace the 5′ portion of the substrate RNA with the 5′ exon of the ribozyme.

These replacement reactions are useful for therapeutic implementations like repairing genetic mutations in mRNAs or insertion of coding sequences of toxic peptides into substrate mRNAs that indicate a disease state.

FIG. 2 illustratively shows a modified group I intron self-splicing system, also called the PIE method. As illustratively shown in FIG. 2, a circularly permuted group I intron precursor RNA consists of end-to-end fused exons that are flanked by half intron sequences. Using a similar mechanism to trans-ribozymes, the permuted group I intron in the presence of GTP and Mg²⁺ generates circular RNA. Arrow 1 shows circular permutation. Arrow 2 shows a first transesterification. Arrow 3 shows a second transesterification. Arrow 4 shows circularized exon. “E1” represents exon 1. “E2” represents exon 2.

The canonical Anabaena permuted intron-exon (Ana-PIE) method for generating circular RNA first described by Puttaraju et al. (Nucleic Acids Res. 1992 Oct. 25; 20(20): 5357-5364), is for RNA circularization. A recent study has revealed that the extra sequences introduced by the self-splicing intron in this method contribute towards immunogenicity of the circRNA products, even after thorough RNA purification (Liu, C. X. et al. RNA circles with minimized immunogenicity as potent PKR inhibitors. Mol. Cell. 82, 420-434 (2022).).

### SUMMARY

In one aspect, the present disclosure provides a method of making circularization of an RNA of interest without a use of permuted intron-exon (PIE), the method including: providing a DNA (deoxyribonucleic acid) construct, wherein the DNA construct includes a Group I intron-based ribozyme, a sequence of interest, and a target sequence; causing an RNA to be transcribed from the DNA construct, the RNA includes a segment S1 corresponding to the Internal guide sequence (IGS) of the ribozyme, the ribozyme, a segment S2 corresponding to the target sequence, and a segment S3 corresponding to the sequence of interest; and effecting a base-pairing between the segment S1 and the segment S2, wherein an occurrence of the base-pairing induces circularization of the segment S3.

In certain embodiment(s), the circularization of the segment S3 includes: moving segment S1 to a 5′ end of the segment S3 and keeping the segment S2 at a 3′ end of the RNA, to generate a scarless circular RNA.

In certain embodiment(s), the base-pairing between the segment S1 and segment S2 includes a wobble base pairing G:U.

In certain embodiment(s), the Group I intron-based ribozyme is a Tetrahymena ribozyme or a Twort ribozyme.

In certain embodiment(s), the Group I intron-based ribozyme is a variant of Tetrahymena.

In certain embodiment(s), the segment S1 or the segment S2 is GNNNNN or NNNNNU, G is guanine, U is uracil, N is any one of adenine, guanine, cytosine, and uracil.

In certain embodiment(s), the segment S1 or the segment S2 is GGAGGG or CCCUCU, G is guanine, U is uracil, A is adenine, and C is cytosine.

In certain embodiment(s), the Group I intron-based ribozyme is Twort, and the segment S1 or the segment S2 is GNNN or NNNU, G is guanine, U is uracil, N is any one of adenine, guanine, cytosine, and uracil.

In certain embodiment(s), the Group I intron-based ribozyme is Twort, and the segment S1 or the segment S2 is GAGC or GCUU, G is guanine, U is uracil, A is adenine, and C is cytosine.

In certain embodiment(s), the method further includes: detecting a circular RNA of the RNA as transcribed in a PCR (polymerase chain reaction) using both a pair of divergent primers and a pair of convergent primers, wherein the pair of convergent primers are oriented in opposite directions (5′ to 3′ and 3′ to 5″) and allow an amplification to proceed inwardly towards each other, wherein the pair of divergent primers are oriented in a same direction (both from 5′ to 3′ or both 3′ to 5′) and allow an amplification to proceed outwardly away from each other.

In another aspect, the present disclosure provides an isolated nucleic acid, the isolated nucleic acid including: a nucleic acid sequence with at least 80% (80 percent) similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21, wherein the at least 80% similarity refers to that at least 8 nucleotides are the same for every 10 nucleotides between two sequences, in a 5′ to 3′ direction.

In yet another aspect, the present disclosure provides a method of forming circular RNA (ribonucleic acid), the method including: providing a DNA (deoxyribonucleic acid) construct including a ribozyme sequence, wherein an RNA segment transcribed from the ribozyme sequence includes a nucleic acid sequence with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21, wherein the at least 80% similarity refers to that at least 8 nucleotides are the same for every 10 nucleotides between two sequences, in a 5′ to 3′ direction.

### BRIEF DESCRIPTION OF THE DRAWINGS

FIG. 1A is a schematic diagram, showing a mechanism of cis-splicing activity of the ribozyme.

FIG. 1B is a schematic diagram, showing a mechanism of trans-splicing activity of the ribozyme.

FIG. 2 is a schematic diagram, showing a mechanism of generating circular RNA by a PIE method.

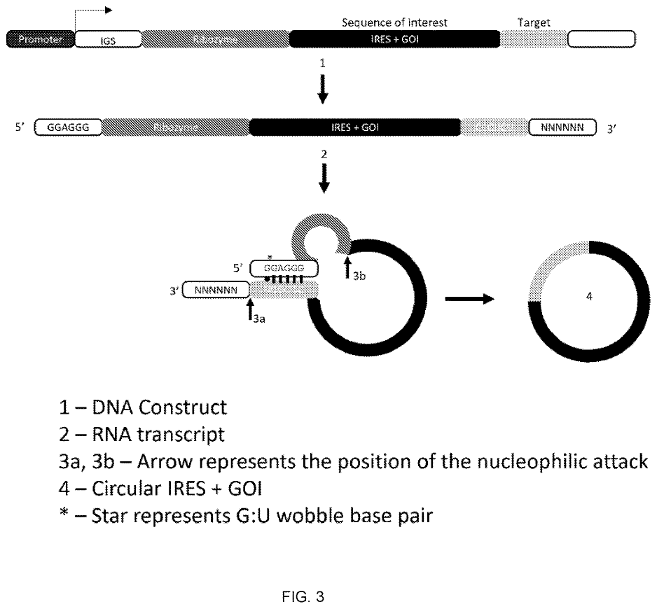

FIG. 3 is a schematic diagram, showing a mechanism of RNA circularization according to certain embodiment(s) of the present disclosure.

FIG. 4 is a schematic diagram, showing a sequence and secondary structure of a WT (wild-type) trans-splicing ribozyme from Tetrahymena thermophilia.

FIG. 5A and FIG. 5B are each a visualization of linearized plasmid DNA after 0.8% agarose gel electrophoresis, according to certain embodiment(s) of the present disclosure.

FIG. 6A and FIG. 6B are each a visualization of RNA generated after IVT for the designed constructs, according to certain embodiment(s) of the present disclosure.

FIG. 7A and FIG. 7B are each a visualization of PCR products after PCR with convergent and divergent primer pairs on 1% agarose gel, according to certain embodiment(s) of the present disclosure.

FIG. 8 is a visualization of RNase R treated RNA on 4% urea-PAGE of RNA113 and RNA114, according to certain embodiment(s) of the present disclosure.

FIG. 9 is a schematic diagram, showing standard curve plots of Ct values against dilution of cDNA for RNA113 and RNA117, according to certain embodiment(s) of the present disclosure.

FIG. 10 is a schematic diagram, showing an alignment of the designed sequence and sequenced reads for RNA 113 and 114, respectively, according to certain embodiment(s) of the present disclosure.

FIG. 11 is a schematic diagram, showing a vector map of SBP113 (wild-type), according to certain embodiment(s) of the present disclosure.

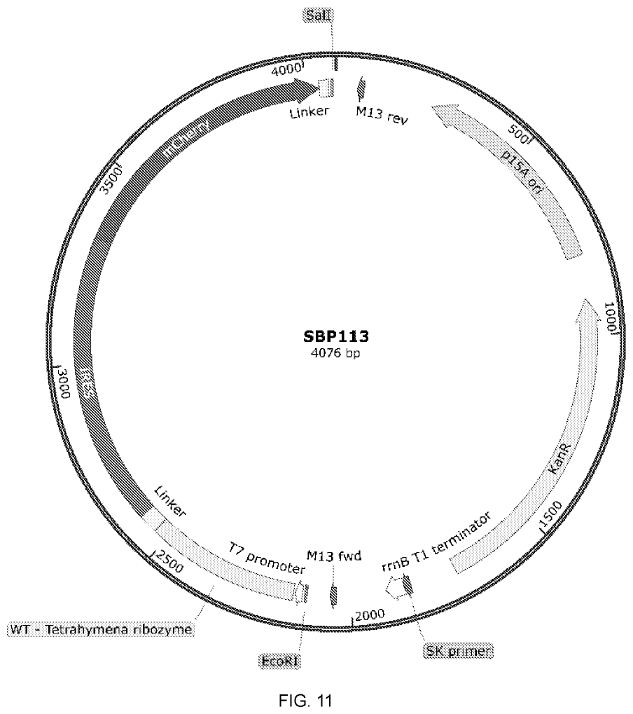

FIG. 12 is a schematic diagram, showing a vector map of SBP114, according to certain embodiment(s) of the present disclosure.

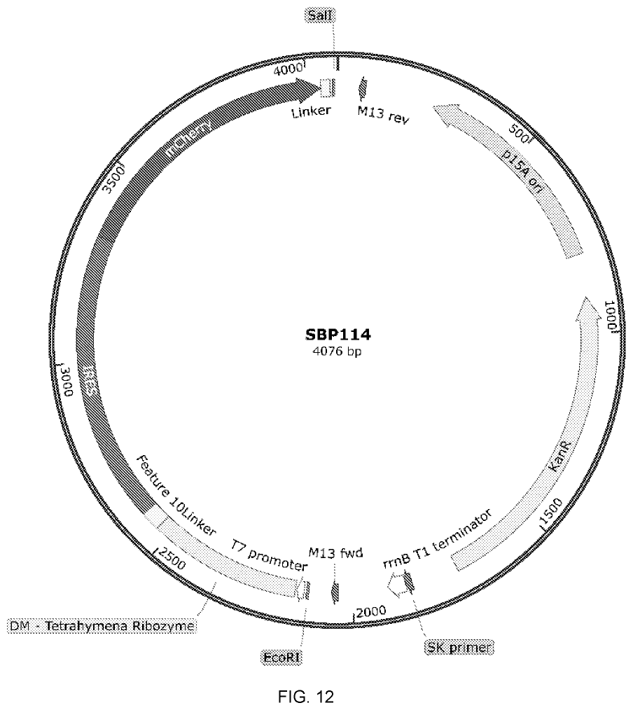

FIG. 13 is a schematic diagram, showing a vector map of SBP115, according to certain embodiment(s) of the present disclosure.

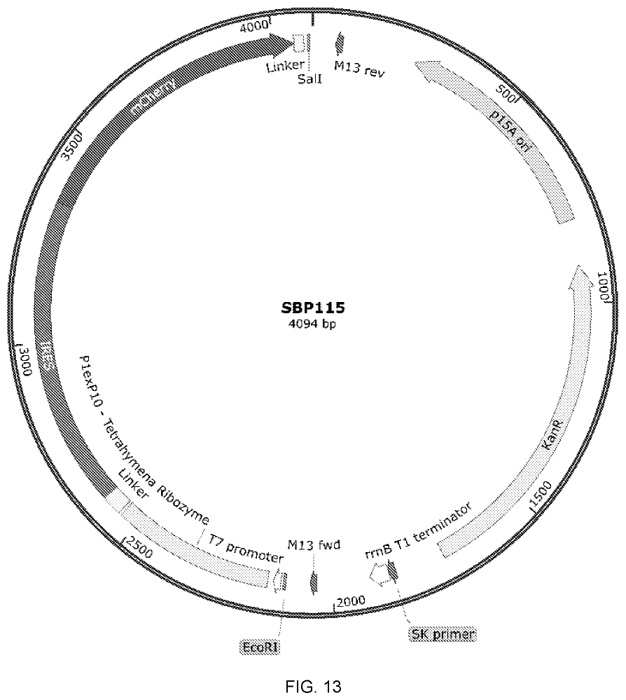

FIG. 14 is a schematic diagram, showing a vector map of SBP116, according to certain embodiment(s) of the present disclosure.

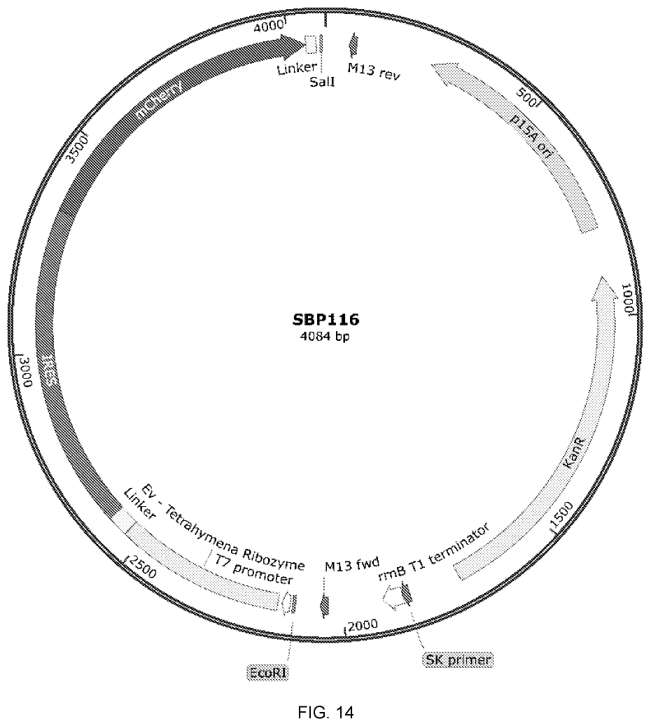

FIG. 15 is a schematic diagram, showing a vector map of SBP117, according to certain embodiment(s) of the present disclosure.

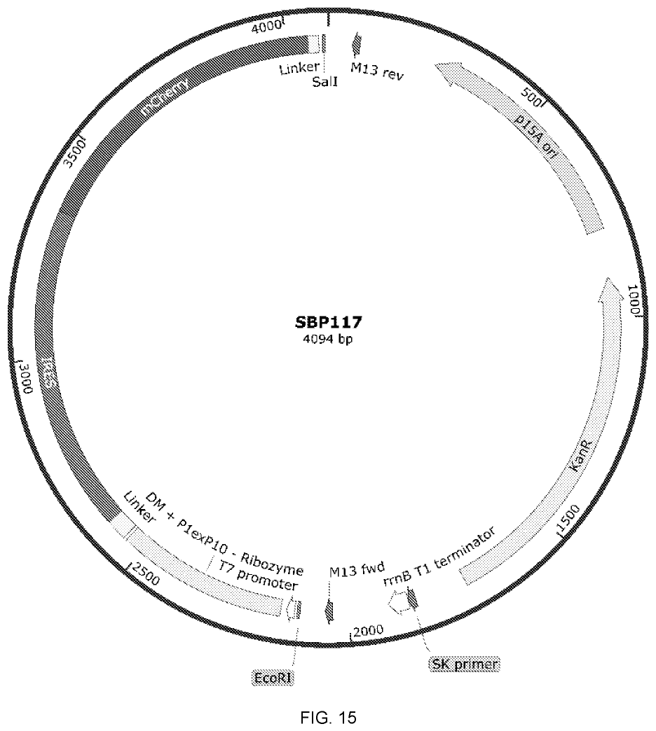

FIG. 16 is a schematic diagram, showing a vector map of SBP118, according to certain embodiment(s) of the present disclosure.

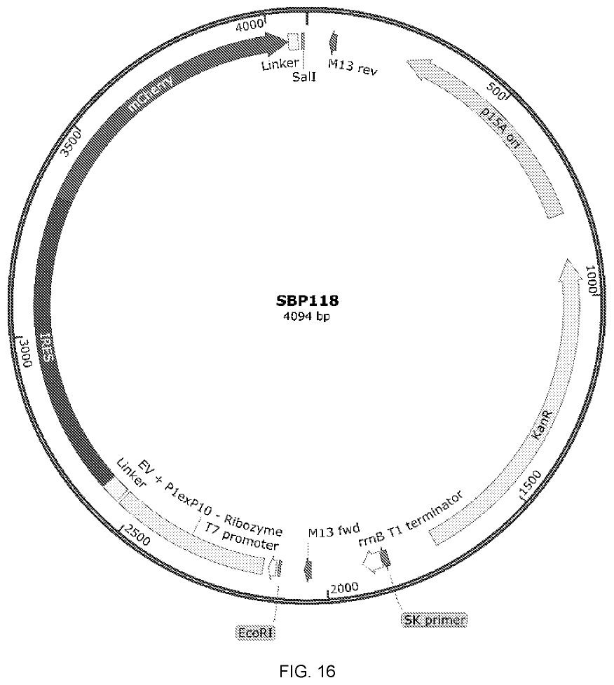

FIG. 17 is a schematic diagram, showing a vector map of SBP119, according to certain embodiment(s) of the present disclosure.

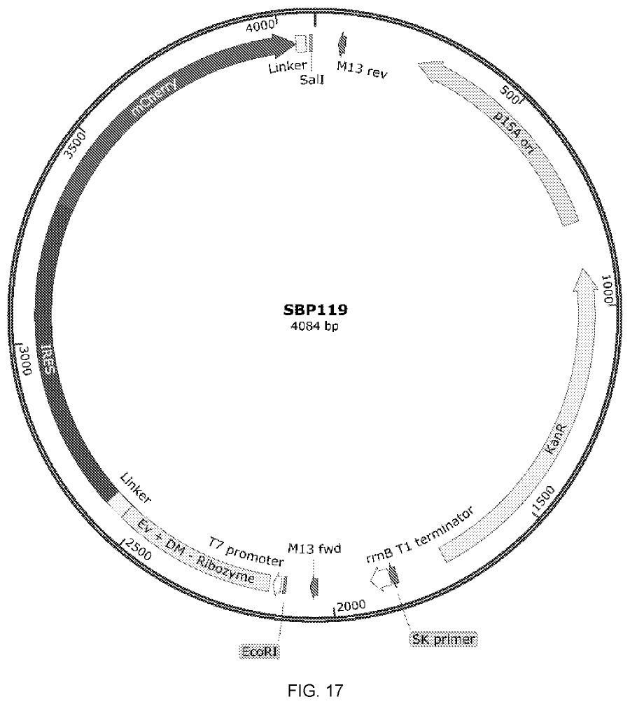

FIG. 18 is a schematic diagram, showing a vector map of SBP120, according to certain embodiment(s) of the present disclosure.

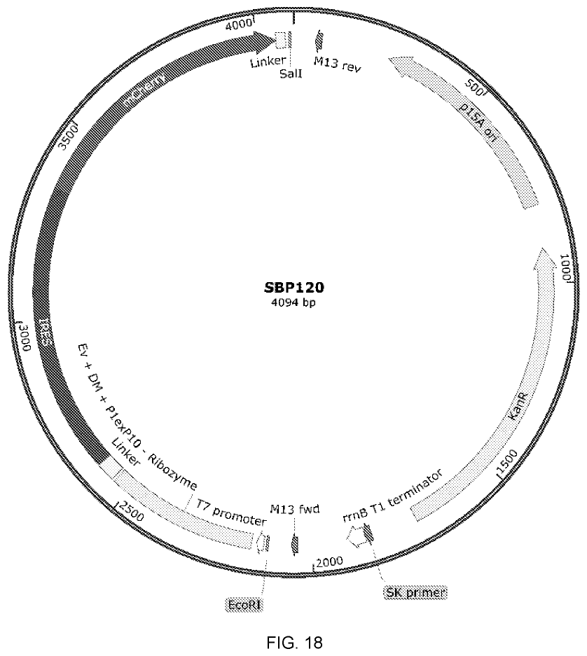

FIG. 19 is a schematic diagram, showing a vector map of SBP121, according to certain embodiment(s) of the present disclosure.

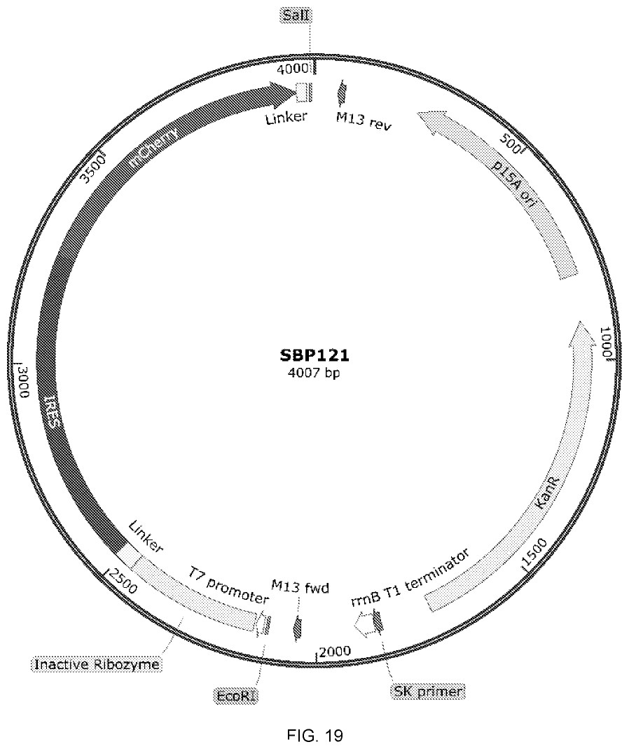

FIG. 20 is a schematic representation of the P1 loop for ribozymes from different origins for which self-splicing ability has been demonstrated.

FIG. 21 is a schematic visualization of RNA after staining on a 4% polyacrylamide-8M Urea gel, where RNA from the sampled circular RNA expression plasmids is generated by IVT.

FIG. 22 is a schematic visualization of RNA samples from RNase R treatment after staining on 4% polyacrylamide-8M urea gel.

FIG. 23 is a schematic mechanism describing a procedure of RT-PCR using convergent and divergent primer pairs.

FIG. 24 is a schematic visualization of RT_PCR products obtained from convergent and divergent primer pairs on a 0.8% agarose gel.

FIG. 25 is a schematic chromatogram from Sanger sequencing of PCR products.

FIG. 26 is a schematic visualization of RNA after staining on a 4% polyacrylamide-8M urea gel.

FIG. 27 is a schematic visualization of RNA samples from RNase R treatment after staining on 4% polyacrylamide-8M urea gel.

FIG. 28 is a schematic diagram of the RNA construct and the generated circular RNA.

FIG. 29 is a schematic visualization of RNA after staining on a 4% polyacrylamide-8M urea gel.

### DETAILED DESCRIPTION

In view of the description of embodiments of the present disclosure in conjunction with the accompanying drawings, aspects and advantages of the present disclosure become readily apparent to those skilled in the art.

In certain embodiment(s), the term “nucleic acid” refers to a polymeric molecule consisting of nucleotide monomers, which may be either deoxyribonucleic acid (DNA) or ribonucleic acid (RNA). DNA and RNA are composed of four types of nucleotide bases namely, adenine (A), guanine (G), cytosine (C), and thymine (T) or uracil (U), respectively. In DNA, these bases are attached to a deoxyribose sugar-phosphate backbone, while in RNA, they are attached to a ribose sugar-phosphate backbone. Nucleic acids carry genetic information and have functional roles in various biological processes, such as gene expression, RNA processing, and catalysis.

In certain embodiment(s) of the present disclosure, the term “plasmid” refers to a circular DNA strand that replicates in the cell independently of the chromosomes.

In certain embodiment(s) of the present disclosure, the term “intron” refers to the nucleotide sequences of a non-protein-coding region of a gene found between two protein-coding regions or exons.

In certain embodiment(s) of the present disclosure, the term “internal guide sequence” or IGS refers to a part of the ribozyme, a polynucleotide sequence near the 5′-end of group I introns that pairs with the target sequence located at the 3′ end of the construct.

In certain embodiment(s) of the present disclosure, the term “ribozyme” refers to a ribonucleic acid enzyme that catalyzes a chemical reaction.

In certain embodiment(s) of the present disclosure, the term “linear RNA” refers to a circRNA precursor capable of forming a circRNA by cyclization reaction.

In certain embodiment(s) of the present disclosure, the term “sequence of interest” or “gene of interest” or “GOI” refers to the polynucleotide sequence within the circRNA that contains the open reading frame (ORF) containing the codons required for expressing the protein of interest after translation, and the regulatory elements required for protein translation such as Internal Ribozyme Entry Site (IRES).

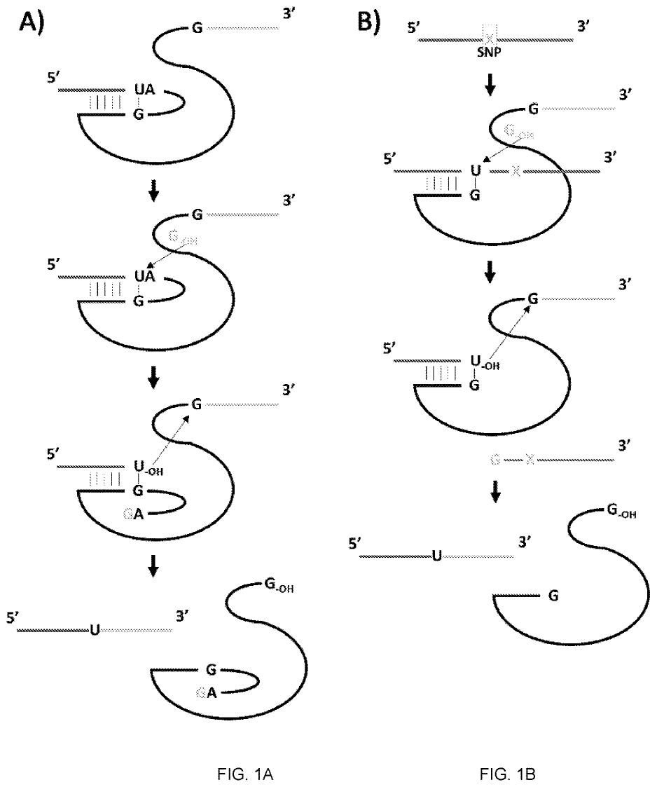

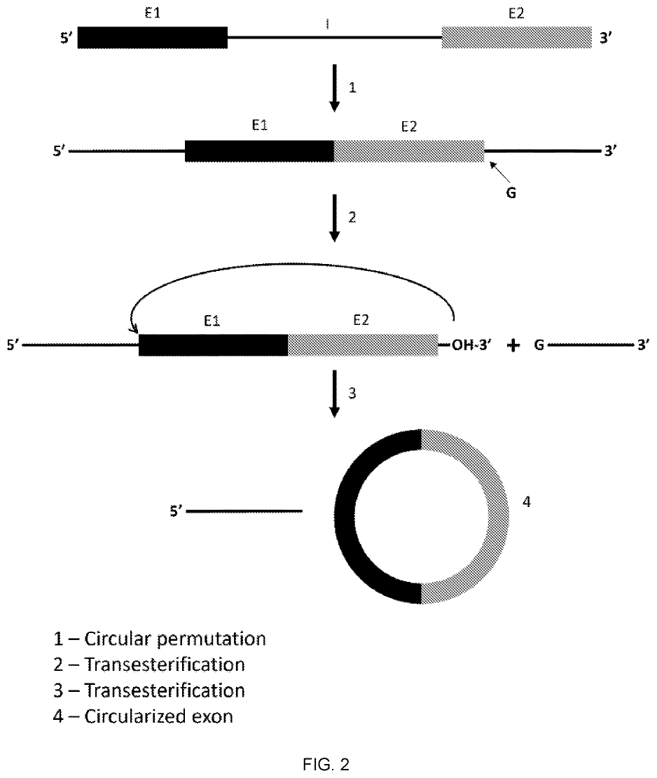

The present disclosure in certain embodiment(s) provides a method for generating scarless RNA (leaves no extraneous sequence at the RNA junction) in a circular form with reduced immunogenicity. In certain embodiment(s), the extraneous sequence refers to a sequence introduced by the self-splicing intron such as the intron presented in FIG. 1A. In certain embodiment(s), plasmids having Tetrahymena ribozyme or Twort ribozyme or one or more of their respective variants without permutation of intron and exon are used in a method to prepare such circular RNA.

In a first aspect, the present disclosure provides an isolated nucleic acid. In certain embodiment(s), the isolated nucleic acid includes a nucleic acid sequence with at least 80% (80 percent) similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 10, SEQ ID NO. 11, SEQ ID NO. 12, SEQ ID NO. 13, or SEQ ID NO. 14, wherein the at least 80% similarity refers to that at least 8 nucleotides are the same for every 10 nucleotides between two sequences, in a 5′ to 3′ direction.

In certain embodiment(s), SEQ ID 01 (as submitted herewith in the Sequence Listing) is of length 393, of molecular type of RNA, of organism Tetrahymena, is referred to “RNA113,” and is of the following sequence structure.

| | |
|---|---|
| ggagggaaaa gttatcaggc atgcacctgg tagctagtct ttaaaccaat agattgcatc | 60 |
| ggtttaaaag gcaagaccgt caaattgcgg gaaaggggtc aacagccgtt cagtaccaag | 120 |
| tctcagggga aactttgaga tggccttgca aagggtatgg taataagctg acggacatgg | 180 |
| tcctaaccac gcagccaagt cctaagtcaa cagatcttct gttgatatgg atgcagttca | 240 |
| cagactaaat gtcggtcggg gaagatgtat tcttctcata agatatagtc ggacctctcc | 300 |
| ttaatgggag ctagcggatg aagtgatgca acactggagc cgctgggaac taatttgtat | 360 |
| gcgaaagtat attgattagt tttggagtac tcg | 393 |

In certain embodiment(s), SEQ ID 02 (as submitted herewith in the Sequence Listing) is of length 393, of molecular type of RNA, of synthetic construct, is referred to “RNA114,” and is of the following sequence structure.

| | |
|---|---|
| ggagggaaaa gttatcaggc atgcacctgg tagctagtct ttaaaccaat agattgcatc | 60 |
| ggtttaaaag gcaagaccgc caaattgcgg gaaaggggtc aacagccgtt cagtaccaag | 120 |
| tctcagggga aactttgaga tggccttgca aagggtatgg taataagctg acggacatgg | 180 |
| tcctaaccac gcagccaagt cctaagtcaa cagatcttct gttgatatgg atgcagttca | 240 |
| cagactaaat ggcggtcggg gaagatgtat tcttctcata agatatagtc ggacctctcc | 300 |
| ttaatgggag ctagcggatg aagtgatgca acactggagc cgctgggaac taatttgtat | 360 |
| gcgaaagtat attgattagt tttggagtac tcg | 393 |

In certain embodiment(s), SEQ ID 03 (as submitted herewith in the Sequence Listing) is of length 402, of molecular type of RNA, of synthetic construct, is referred to “RNA115,” and is of the following sequence structure.

| | |
|---|---|
| ggtacctttg gagggaaaag ttatcaggca tgcacctggt agctagtctt taaaccaata | 60 |
| gattgcatcg gtttaaaagg caagaccgtc aaattgcggg aaaggggtca acagccgttc | 120 |
| agtaccaagt ctcaggggaa actttgagat ggccttgcaa agggtatggt aataagctga | 180 |
| cggacatggt cctaaccacg cagccaagtc ctaagtcaac agatcttctg ttgatatgga | 240 |
| tgcagttcac agactaaatg tcggtcgggg aagatgtatt cttctcataa gatatagtcg | 300 |
| gacctctcct taatgggagc tagcggatga agtgatgcaa cactggagcc gctgggaact | 360 |
| aatttgtatg cgaaagtata ttgattagtt ttggagtact cg | 402 |

In certain embodiment(s), SEQ ID 04 (as submitted herewith in the Sequence Listing) is of length 398, of molecular type of RNA, of synthetic construct, is referred to “RNA116,” and is of the following sequence structure.

| | |
|---|---|
| ggtctggagg gaaaagttat caggcatgca cctggtagct agtctttaaa ccaatagatt | 60 |
| gcatcggttt aaaaggcaag accgtcaaat tgcgggaaag gggtcaacag ccgttcagta | 120 |
| ccaagtctca ggggaaactt tgagatggcc ttgcaaaggg tatggtaata agctgacgga | 180 |
| catggtccta accacgcagc caagtcctaa gtcaacagat cttctgttga tatggatgca | 240 |
| gttcacagac taaatgtcgg tcggggaaga tgtattcttc tcataagata tagtcggacc | 300 |
| tctccttaat gggagctagc ggatgaagtg atgcaacact ggagccgctg ggaactaatc | 360 |
| tgtatgcgaa agtatattga ttagttttgg agtactcg | 398 |

In certain embodiment(s), SEQ ID 05 (as submitted herewith in the Sequence Listing) is of length 402, of molecular type of RNA, of synthetic construct, is referred to “RNA117,” and is of the following sequence structure.

| | |
|---|---|
| ggtacctttg gagggaaaag ttatcaggca tgcacctggt agctagtctt taaaccaata | 60 |
| gattgcatcg gtttaaaagg caagaccgcc aaattgcggg aaaggggtca acagccgttc | 120 |
| agtaccaagt ctcaggggaa actttgagat ggccttgcaa agggtatggt aataagctga | 180 |
| cggacatggt cctaaccacg cagccaagtc ctaagtcaac agatcttctg ttgatatgga | 240 |
| tgcagttcac agactaaatg gcggtcgggg aagatgtatt cttctcataa gatatagtcg | 300 |
| gacctctcct taatgggagc tagcggatga agtgatgcaa cactggagcc gctgggaact | 360 |
| aatttgtatg cgaaagtata ttgattagtt ttggagtact cg | 402 |

In certain embodiment(s), SEQ ID 06 (as submitted herewith in the Sequence Listing) is of length 402, of molecular type of RNA, of synthetic construct, is referred to “RNA118,” and is of the following sequence structure.

| | |
|---|---|
| ggtacctctg gagggaaaag ttatcaggca tgcacctggt agctagtctt taaaccaata | 60 |
| gattgcatcg gtttaaaagg caagaccgtc aaattgggg aaaggggtca acagccgttc | 120 |
| agtaccaagt ctcaggggaa actttgagat ggccttgcaa agggtatggt aataagctga | 180 |
| cggacatggt cctaaccacg cagccaagtc ctaagtcaac agatcttctg ttgatatgga | 240 |
| tgcagttcac agactaaatg tcggtcgggg aagatgtatt cttctcataa gatatagtcg | 300 |
| gacctctcct taatgggagc tagcggatga agtgatgcaa cactggagcc gctgggaact | 360 |
| aatctgtatq cgaaagtata ttgattagtt ttqgagtact ca | 402 |

In certain embodiment(s), SEQ ID 07 (as submitted herewith in the Sequence Listing) is of length 398, of molecular type of RNA, of synthetic construct, is referred to “RNA119,” and is of the following sequence structure.

| | |
|---|---|
| ggtctggagg gaaaagttat caggcatgca cctggtagct agtctttaaa ccaatagatt | 60 |
| gcatcggttt aaaaggcaag accgccaaat tgcgggaaag gggtcaacag ccgttcagta | 120 |
| ccaagtctca ggggaaactt tgagatggcc ttgcaaaggg tatggtaata agctgacgga | 180 |
| catggtccta accacgcagc caagtcctaa gtcaacagat cttctgttga tatggatgca | 240 |
| gttcacagac taaatggcgg tcggggaaga tgtattcttc tcataagata tagtcggacc | 300 |
| tctccttaat gggagctagc ggatgaagtg atgcaacact ggagccgctg ggaactaatc | 360 |
| tgtatgcgaa agtatattga ttagttttgg agtactcg | 398 |

In certain embodiment(s), SEQ ID 08 (as submitted herewith in the Sequence Listing) is of length 402, of molecular type of RNA, of synthetic construct, is referred to “RNA120,” and is of the following sequence structure.

| | |
|---|---|
| ggtacctctg gagggaaaag ttatcaggca tgcacctggt agctagtctt taaaccaata | 60 |
| gattgcatcg gtttaaaagg caagaccgcc aaattgcggg aaaggggtca acagccgttc | 120 |
| agtaccaagt ctcaggggaa actttgagat ggccttgcaa agggtatggt aataagctga | 180 |
| cggacatggt cctaaccacg cagccaagtc ctaagtcaac agatcttctg ttgatatgga | 240 |
| tgcagttcac agactaaatg gcggtcgggg aagatgtatt cttctcataa gatatagtcg | 300 |
| gacctctcct taatgggagc tagcggatga agtgatgcaa cactggagcc gctgggaact | 360 |
| aatctgtatg cgaaagtata ttgattagtt ttggagtact cg | 402 |

In certain embodiment(s), SEQ ID 09 (as submitted herewith in the Sequence Listing) is of length 324, of molecular type of RNA, of synthetic construct, is referred to “RNA121,” and is of the following sequence structure.

| | |
|---|---|
| ggagggaaaa gttatcaggc atgcacctgg tagctagtct ttaaaccaat agattgcatc | 60 |
| ggtttaaaag gcaagaccgt caaattgcgg gaaaggggtc aacagacatg gtcctaacca | 120 |
| cgcagccaag tcctaagtca acagatcttc tgttgatatg gatgcagttc acagactaaa | 180 |
| tgtcggtcgg ggaagatgta ttcttctcat aagatatagt cggacctctc cttaatggga | 240 |
| gctagcggat gaagtgatgc aacactggag ccgctgggaa ctaatttgta tgcgaaagta | 300 |
| tattgattag ttttggagta ctcg | 324 |

In certain embodiment(s), SEQ ID 10 (as submitted herewith in the Sequence Listing) is of length 241, of molecular type of RNA, of synthetic construct of organism Anabaena, is referred to “RNA124,” and is of the following sequence structure.

| | |
|---|---|
| gagccttaga gaagaaattc tttaagtgga tgctctcaaa ctcagggaaa cctaaatcta | 60 |
| gctatagaca aggcaatcct gagccaagcc gaagtagtaa ttagtaagtt aacaacagat | 120 |
| aacttacagc tagtcggaag gtgcagagac tcgacgggag ctaccctaac gtcaagacga | 180 |
| gggtaaagag agagtccaat tctcaaagcc aataggcagt agcgaaagct gcgggagaat | 240 |
| g | 241 |

In certain embodiment(s), SEQ ID 11 (as submitted herewith in the Sequence Listing) is of length 197, of molecular type of RNA, of synthetic construct of organism Azoarcus, is referred to “RNA125,” and is of the following sequence structure.

| | |
|---|---|
| gtgccttgcg ccgggaaacc acgcaaggga tggtgtcaaa ttcggcgaaa cctaagcgcc | 60 |
| cgcccgggcg tatggcaacg ccgagccaag cttcggcgcc tgcgccgatg aaggtgtaga | 120 |
| gactagacgg cacccaccta aggcaaacgc tatggtgaag gcatagtcca gggagtggcg | 180 |
| aaagtcacac aaaccgg | 197 |

In certain embodiment(s), SEQ ID 12 (as submitted herewith in the Sequence Listing) is of length 244, of molecular type of RNA, of synthetic construct of organism Twort, is referred to “RNA126,” and is of the following sequence structure.

| | |
|---|---|
| gagcctttat acagtaatgt atatcgaaaa atcctctaat tcagggaaca cctaaacaaa | 60 |
| ctaagatgta ggcaatcctg agctaagctc ttagtaataa gagaaagtgc aacgactatt | 120 |
| ccgataggaa gtagggtcaa gtgactcgaa atggggatta cccttctagg gtagtgatat | 180 |
| agtctgaaca tatatggaaa catatagaag gataggagta acgaacctat ccgtaacata | 240 |
| attg | 244 |

In certain embodiment(s), SEQ ID 13 (as submitted herewith in the Sequence Listing) is of length 382, of molecular type of RNA, of synthetic construct of organism Scytalidium, is referred to “RNA127,” and is of the following sequence structure.

gcctgtgaca gcggggcttc gaacttttcc gtgttttttt
tttttcttcc gctagtcgat catccgtgac ggccgggcaa
gcgcccgagt accaggaccg tcgggccccc gagaaggggg
ggcctaggat gcggcaagac gacccggttc ggggaacgcc
agcgtgcgct ggccgatccc gaggcgaggt gccgtagcgg
gcacccgtcg taacgcgcgg tagggcgtcg gtcccccctc
ccacggcgga ggggaggctt aaggtacgtg ctaaaccccc
agcgatgggg cctgtaggaa aagccctggt acggcgaagc
ctacggggac cgcccgatgg cggtcggatg ccggcgggcc
acggagcccc cggcgtcgat tg

In certain embodiment(s), SEQ ID 14 (as submitted herewith in the Sequence Listing) is of length 279, of molecular type of RNA, of synthetic construct of organism Clostridium, is referred to “RNA128,” and is of the following sequence structure.

gtcgccttat gaagtgatta ctaagtgaaa
acttggcttt atcggtgaaa ccgaaacgta
aagacgtcgg caataccgag tggtatgtgg
ggaaacccaa cagccgtgta tcgactcata
ggtttctaac cttaagaaat gaaattttct
taagtaagaa atactaggat agaagtttta
aactataata tagcttctat aacatgccaa
ggtacttaaa acttataagt tttaagcacg
agatatagtc agtgccatta gagataatgg
aatagcatg

In certain embodiment(s), SEQ ID 15 (as submitted herewith in the Sequence Listing) is of length 19, of molecular type of DNA, of synthetic construct, is referred to “T7 promoter,” and is of the following sequence structure.

taatacgact cactatagg

In certain embodiment(s), SEQ ID 16 (as submitted herewith in the Sequence Listing) is of length 711, of molecular type of DNA, of synthetic construct, is referred to “mCherry” and is of the following sequence structure.

| | |
|---|---|
| atggtcagca agggcgagga agacaatatg gcaatcatca aggagttcat gagattcaag | |
| gttcacatgg aaggttctgt caatgggcac gagttcgaga tcgagggaga gggagaggga | |
| aggccatacg agggaacaca aacagccaag ttaaaggtca cgaagggcgg gcctcttcca | |
| ttcgcatggg acatattaag tcctcaattc atgtacgggt ctaaggcata cgttaagcac | |
| cccgcagaca taccagacta cttgaagctc tccttccccg agggattcaa gtgggagagg | |
| gtcatgaatt tcgaggacgg tggggtagtt acggttactc aagactcttc cttacaagac | |
| ggagagttca tatacaaggt taagttacgg ggcactaatt tccctagtga cgggccagtt | |
| atgcaaaaga agacgatggg atgggaagcc agttcagaga ggatgtaccc agaggacggt | 480 |
| gccttaaagg gagagatcaa gcaacgactc aagttgaagg acggtggaca ctacgacgct | 540 |
| gaggtaaaga cgacgtacaa ggccaagaag ccagtacaac tccccggagc ttacaatgtt | 600 |
| aatatcaagc ttgacatcac ttcccacaat gaggactaca caatcgttga gcaatacgag | |
| cgggccgagg gacggcactc aacaggtgga atggacgagc tctacaagta g | |

In certain embodiment(s), SEQ ID 17 (as submitted herewith in the Sequence Listing) is of length 1959, of molecular type of DNA, of synthetic construct, is referred to “SBP113,” and is of the following sequence structure.

GAATTCCAGCTGTAATACGACTCACTATAGGAGGGAAAAGTTATCA
GGCATGCACCTGGTAGCTAGTCTTTAAACCAATAGATTGCATCGGT
TTAAAAGGCAAGACCGTCAAATTGCGGGAAAGGGGTCAACAGCCG
TTCAGTACCAAGTCTCAGGGGAAACTTTGAGATGGCCTTGCAAAG
GGTATGGTAATAAGCTGACGGACATGGTCCTAACCACGCAGCCAA
GTCCTAAGTCAACAGATCTTCTGTTGATATGGATGCAGTTCACAGA
CTAAATGTCGGTCGGGGAAGATGTATTCTTCTCATAAGATATAGTCG
GACCTCTCCTTAATGGGAGCTAGCGGATGAAGTGATGCAACACTG
GAGCCGCTGGGAACTAATTTGTATGCGAAAGTATATTGATTAGTTTT
GGAGTACTCGTAAAAACAAAAACAAAAACAAAAACCAAAAACAAAA
ACAAAAACACACATTAAAACAGCCTGTGGGTTGATCCCACCCACA
GGCCCATTGGGCGCTAGCACTCTGGTATCACGGTACCTTTGTGCG
CCTGTTTTATACCCCCTCCCCCAACTGTAACTTAGAAGTAACACAC
ACCGATCAACAGTCAGCGTGGCACACCAGCCACGTTTTGATCAAG
CACTTCTGTTACCCCGGACTGAGTATCAATAGACTGCTCACGCGGT
TGAAGGAGAAAGCGTTCGTTATCCGGCCAACTACTTCGAAAAACC
TAGTAACACCGTGGAAGTTGCAGAGTGTTTCGCTCAGCACTACCC
CAGTGTAGATCAGGTCGATGAGTCACCGCATTCCCCACGGGCGAC
CGTGGCGGTGGCTGCGTTGGCGGCCTGCCCATGGGGAAACCCAT
GGGACGCTCTAATACAGACATGGTGCGAAGAGTCTATTGAGCTAGT
TGGTAGTCCTCCGGCCCCTGAATGCGGCTAATCCTAACTGCGGAG
CACACACCCTCAAGCCAGAGGGCAGTGTGTCGTAACGGGCAACT
CTGCAGCGGAACCGACTACTTTGGGTGTCCGTGTTTCATTTTATTC
CTATACTGGCTGCTTATGGTGACAATTGAGAGATCGTTACCATATAG
CTATTGGATTGGCCATCCGGTGACTAATAGAGCTATTATATATCCCTT
TGTTGGGTTTATACCACTTAGCTTGAAAGAGGTTAAAACATTACAAT
TCATTGTTAAGTTGAATACAGCAAAATGGTCAGCAAGGGCGAGGAA
GACAATATGGCAATCATCAAGGAGTTCATGAGATTCAAGGTTCACA
TGGAAGGTTCTGTCAATGGGCACGAGTTCGAGATCGAGGGAGAG
GGAGAGGGAAGGCCATACGAGGGAACACAAACAGCCAAGTTAAA
GGTCACGAAGGGCGGGCCTCTTCCATTCGCATGGGACATATTAAG
TCCTCAATTCATGTACGGGTCTAAGGCATACGTTAAGCACCCCGCA
GACATACCAGACTACTTGAAGCTCTCCTTCCCCGAGGGATTCAAGT
GGGAGAGGGTCATGAATTTCGAGGACGGTGGGGTAGTTACGGTTA
CTCAAGACTCTTCCTTACAAGACGGAGAGTTCATATACAAGGTTAA
GTTACGGGGCACTAATTTCCCTAGTGACGGGCCAGTTATGCAAAA
GAAGACGATGGGATGGGAAGCCAGTTCAGAGAGGATGTACCCAG
AGGACGGTGCCTTAAAGGGAGAGATCAAGCAACGACTCAAGTTGA
AGGACGGTGGACACTACGACGCTGAGGTAAAGACGACGTACAAG
GCCAAGAAGCCAGTACAACTCCCCGGAGCTTACAATGTTAATATCA
AGCTTGACATCACTTCCCACAATGAGGACTACACAATCGTTGAGCA
ATACGAGCGGGCCGAGGGACGGCACTCAACAGGTGGAATGGACG
AGCTCTACAAGTAGAAAAACAAAAACAAAAACAAAAACCCCTCTGT
CGAC

In certain embodiment(s), SEQ ID 18 (as submitted herewith in the Sequence Listing) is of length 1814, of molecular type of DNA, of synthetic construct, is referred to “SBP126,” and is of the following sequence structure.

GAATTCCAGCTGTAATACGACTCACTATAggaaGAGCCTTTATACAGT
AATGTATATCGAAAAATCCTCTAATTCAGGGAACACCTAAACAAACT
AAGATGTAGGCAATCCTGAGCTAAGCTCTTAGTAATAAGAGAAAGT
GCAACGACTATTCCGATAGGAAGTAGGGTCAAGTGACTCGAAATG
GGGATTACCCTTCTAGGGTAGTGATATAGTCTGAACATATATGGAAA
CATATAGAAGGATAGGAGTAACGAACCTATCCGTAACATAATTGaAA
AAACAAAAACAAAAACAAAAACCAAAAACAAAAACAAAAACACACA
TTAAAACAGCCTGTGGGTTGATCCCACCCACAGGCCCATTGGGCG
CTAGCACTCTGGTATCACGGTACCTTTGTGCGCCTGTTTTATACCC
CCTCCCCCAACTGTAACTTAGAAGTAACACACACCGATCAACAGTC
AGCGTGGCACACCAGCCACGTTTTGATCAAGCACTTCTGTTACCC
CGGACTGAGTATCAATAGACTGCTCACGCGGTTGAAGGAGAAAGC
GTTCGTTATCCGGCCAACTACTTCGAAAAACCTAGTAACACCGTGG
AAGTTGCAGAGTGTTTCGCTCAGCACTACCCCAGTGTAGATCAGG
TCGATGAGTCACCGCATTCCCCACGGGCGACCGTGGCGGTGGCT
GCGTTGGCGGCCTGCCCATGGGGAAACCCATGGGACGCTCTAATA
CAGACATGGTGCGAAGAGTCTATTGAGCTAGTTGGTAGTCCTCCG
GCCCCTGAATGCGGCTAATCCTAACTGCGGAGCACACACCCTCAA
GCCAGAGGGCAGTGTGTCGTAACGGGCAACTCTGCAGCGGAACC
GACTACTTTGGGTGTCCGTGTTTCATTTTATTCCTATACTGGCTGCT
TATGGTGACAATTGAGAGATCGTTACCATATAGCTATTGGATTGGCC
ATCCGGTGACTAATAGAGCTATTATATATCCCTTTGTTGGGTTTATAC
CACTTAGCTTGAAAGAGGTTAAAACATTACAATTCATTGTTAAGTTG
AATACAGCAAAATGGTCAGCAAGGGCGAGGAAGACAATATGGCAA
TCATCAAGGAGTTCATGAGATTCAAGGTTCACATGGAAGGTTCTGT
CAATGGGCACGAGTTCGAGATCGAGGGAGAGGGAGAGGGAAGG
CCATACGAGGGAACACAAACAGCCAAGTTAAAGGTCACGAAGGGC
GGGCCTCTTCCATTCGCATGGGACATATTAAGTCCTCAATTCATGTA
CGGGTCTAAGGCATACGTTAAGCACCCCGCAGACATACCAGACTA
CTTGAAGCTCTCCTTCCCCGAGGGATTCAAGTGGGAGAGGGTCAT
GAATTTCGAGGACGGTGGGGTAGTTACGGTTACTCAAGACTCTTC
CTTACAAGACGGAGAGTTCATATACAAGGTTAAGTTACGGGGCACT
AATTTCCCTAGTGACGGGCCAGTTATGCAAAAGAAGACGATGGGAT
GGGAAGCCAGTTCAGAGAGGATGTACCCAGAGGACGGTGCCTTA
AAGGGAGAGATCAAGCAACGACTCAAGTTGAAGGACGGTGGACA
CTACGACGCTGAGGTAAAGACGACGTACAAGGCCAAGAAGCCAGT
ACAACTCCCCGGAGCTTACAATGTTAATATCAAGCTTGACATCACTT
CCCACAATGAGGACTACACAATCGTTGAGCAATACGAGCGGGCCG
AGGGACGGCACTCAACAGGTGGAATGGACGAGCTCTACAAGTAG
AAAAACAAAAACAAAAACAAAAACGCTTTTGTCGAC

In certain embodiment(s), SEQ ID 19 (as submitted herewith in the Sequence Listing) is of length 741, of molecular type of DNA, of synthetic construct, is referred to “CVB3 IRES” or “IRES,” and is of the following sequence structure.

ttaaaacagc ctgtgggttg atcccaccca caggcccatt
gggcgctagc actctggtat cacggtacct ttgtgcgcct
gttttatacc ccctccccca actgtaactt agaagtaaca
cacaccgatc aacagtcagc gtggcacacc agccacgttt
tgatcaagca cttctgttac cccggactga gtatcaatag
actgctcacg cggttgaagg agaaagcgtt cgttatccgg
ccaactactt cgaaaaacct agtaacaccg tggaagttgc
agagtgtttc gctcagcact accccagtgt agatcaggtc
gatgagtcac cgcattcccc acgggcgacc gtggcggtgg
ctgcgttggc ggcctgccca tggggaaacc catgggacgc
tctaatacag acatggtgcg aagagtctat tgagctagtt
ggtagtcctc cggcccctga atgcggctaa tcctaactgc
ggagcacaca ccctcaagcc agagggcagt gtgtcgtaac
gggcaactct gcagcggaac cgactacttt gggtgtccgt
gtttcatttt attcctatac tggctgctta tggtgacaat
tgagagatcg ttaccatata gctattggat tggccatccg
gtgactaata gagctattat atatcccttt gttgggttta
taccacttag cttgaaagag gttaaaacat tacaattcat
tgttaagttg aatacagcaa a

In certain embodiment(s), SEQ ID 20 (as submitted herewith in the Sequence Listing) is of length 1922, of molecular type of RNA, of synthetic construct, is referred to “RNA134,” and is of the following sequence structure.

GGAGGGAAAAGTTATCAGGCATGCACCTGGTAGCTAGTCTTTAAACCAAT
AGATTGCATCGGTTTAAAAGGCAAGACCGTCAAATTGCGGGAAAGGGGTC
AACAGCCGTTCAGTACCAAGTCTCAGGGGAAACTTTGAGATGGCCTTGCA
AAGGGTATGGTAATAAGCTGACGGACATGGTCCTAACCACGCAGCCAAGT
CCTAAGTCAACAGATCTTCTGTTGATATGGATGCAGTTCACAGACTAAAT
GTCGGTCGGGGAAGATGTATTCTTCTCATAAGATATAGTCGGACCTCTCC
TTAATGGGAGCTAGCGGATGAAGTGATGCAACACTGGAGCCGCTGGGAAC
TAATTTGTATGCGAAAGTATATTGATTAGTTTTGGAGTACTCGTCCATTC
GCATGGGACATATTAAGTCCTCAATTCATGTACGGGTCTAAGGCATACGT
TAAGCACCCCGCAGACATACCAGACTACTTGAAGCTCTCCTTCCCCGAGG
GATTCAAGTGGGAGAGGGTCATGAATTTCGAGGACGGTGGGGTAGTTACG
GTTACTCAAGACTCTTCCTTACAAGACGGAGAGTTCATATACAAGGTTAA
GTTACGGGGCACTAATTTCCCTAGTGACGGGCCAGTTATGCAAAAGAAGA
CGATGGGATGGGAAGCCAGTTCAGAGAGGATGTACCCAGAGGACGGTGCC
TTAAAGGGAGAGATCAAGCAACGACTCAAGTTGAAGGACGGTGGACACTA
CGACGCTGAGGTAAAGACGACGTACAAGGCCAAGAAGCCAGTACAACTCC
CCGGAGCTTACAATGTTAATATCAAGCTTGACATCACTTCCCACAATGAG
GACTACACAATCGTTGAGCAATACGAGCGGGCCGAGGGACGGCACTCAAC
AGGTGGAATGGACGAGCTCTACAAGTAGAAAAACAAAAACAAAAACAAAA
ACAAAAACAAAAACAAAAACAAAAACCAAAAACAAAAACAAAAACACACA
TTAAAACAGCCTGTGGGTTGATCCCACCCACAGGCCCATTGGGCGCTAGC
ACTCTGGTATCACGGTACCTTTGTGCGCCTGTTTTATACCCCCTCCCCCA
ACTGTAACTTAGAAGTAACACACACCGATCAACAGTCAGCGTGGCACACC
AGCCACGTTTTGATCAAGCACTTCTGTTACCCCGGACTGAGTATCAATAG
TACTTCGAAAAACCTAGTAACACCGTGGAAGTTGCAGAGTGTTTCGCTCA
ACTGCTCACGCGGTTGAAGGAGAAAGCGTTCGTTATCCGGCCAACGCACT
ACCCCAGTGTAGATCAGGTCGATGAGTCACCGCATTCCCCACGGGCGACC
GTGGCGGTGGCTGCGTTGGCGGCCTGCCCATGGGGAAACCCATGGGACGC
TCTAATACAGACATGGTGCGAAGAGTCTATTGAGCTAGTTGGTAGTCCTC
CGGCCCCTGAATGCGGCTAATCCTAACTGCGGAGCACACACCCTCAAGCC
AGAGGGCAGTGTGTCGTAACGGGCAACTCTGCAGCGGAACCGACTACTTT
GGGTGTCCGTGTTTCATTTTATTCCTATACTGGCTGCTTATGGTGACAAT
TGAGAGATCGTTACCATATAGCTATTGGATTGGCCATCCGGTGACTAATA
GAGCTATTATATATCCCTTTGTTGGGTTTATACCACTTAGCTTGAAAGAG
GTTAAAACATTACAATTCATTGTTAAGTTGAATACAGCAAAATGGTCAGC
AAGGGCGAGGAAGACAATATGGCAATCATCAAGGAGTTCATGAGATTCAA
GGTTCACATGGAAGGTTCTGTCAATGGGCACGAGTTCGAGATCGAGGGAG
AGGGAGAGGGAAGGCCATACGAGGGAACACAAACAGCCAAGTTAAAGGTC
ACGAAGGGCGGCCCTCTGTCGA

In certain embodiment(s), SEQ ID 21 (as submitted herewith in the Sequence Listing) is of length 1780, of molecular type of RNA, of synthetic construct, is referred to “RNA138,” and is of the following sequence structure.

GGAAGAGCCTTTATACAGTAATGTATATCGAAAAATCCTCTAATTCAGGG
AACACCTAAACAAACTAAGATGTAGGCAATCCTGAGCTAAGCTCTTAGTA
ATAAGAGAAAGTGCAACGACTATTCCGATAGGAAGTAGGGTCAAGTGACT
CGAAATGGGGATTACCCTTCTAGGGTAGTGATATAGTCTGAACATATATG
GAAACATATAGAAGGATAGGAGTAACGAACCTATCCGTAACATAATTGAC
AATGTTAATATCAAGCTTGACATCACTTCCCACAATGAGGACTACACAAT
CGTTGAGCAATACGAGCGGGCCGAGGGACGGCACTCAACAGGTGGAATGG
ACGAGCTCTACAAGTAGAAAAACAAAAACAAAAACAAAAACaAAAAACAA
AAACAAAAACAAAAACCAAAAACAAAAACAAAAACACACATTAAAACAGC
CTGTGGGTTGATCCCACCCACAGGCCCATTGGGCGCTAGCACTCTGGTAT
CACGGTACCTTTGTGCGCCTGTTTTATACCCCCTCCCCCAACTGTAACTT
AGAAGTAACACACACCGATCAACAGTCAGCGTGGCACACCAGCCACGTTT
TGATCAAGCACTTCTGTTACCCCGGACTGAGTATCAATAGACTGCTCACG
CGGTTGAAGGAGAAAGCGTTCGTTATCCGGCCAACTACTTCGAAAAACCT
AGTAACACCGTGGAAGTTGCAGAGTGTTTCGCTCAGCACTACCCCAGTGT
AGATCAGGTCGATGAGTCACCGCATTCCCCACGGGCGACCGTGGCGGTGG
CTGCGTTGGCGGCCTGCCCATGGGGAAACCCATGGGACGCTCTAATACAG
ACATGGTGCGAAGAGTCTATTGAGCTAGTTGGTAGTCCTCCGGCCCCTGA
ATGCGGCTAATCCTAACTGCGGAGCACACACCCTCAAGCCAGAGGGCAGT
GTGTCGTAACGGGCAACTCTGCAGCGGAACCGACTACTTTGGGTGTCCGT
GTTTCATTTTATTCCTATACTGGCTGCTTATGGTGACAATTGAGAGATCG
TTACCATATAGCTATTGGATTGGCCATCCGGTGACTAATAGAGCTATTAT
ATATCCCTTTGTTGGGTTTATACCACTTAGCTTGAAAGAGGTTAAAACAT
TACAATTCATTGTTAAGTTGAATACAGCAAAATGGTCAGCAAGGGCGAGG
AAGACAATATGGCAATCATCAAGGAGTTCATGAGATTCAAGGTTCACATG
GAAGGTTCTGTCAATGGGCACGAGTTCGAGATCGAGGGAGAGGGAGAGGG
AAGGCCATACGAGGGAACACAAACAGCCAAGTTAAAGGTCACGAAGGGCG
GGCCTCTTCCATTCGCATGGGACATATTAAGTCCTCAATTCATGTACGGG
TCTAAGGCATACGTTAAGCACCCCGCAGACATACCAGACTACTTGAAGCT
CTCCTTCCCCGAGGGATTCAAGTGGGAGAGGGTCATGAATTTCGAGGACG
GTGGGGTAGTTACGGTTACTCAAGACTCTTCCTTACAAGACGGAGAGTTC
ATATACAAGGTTAAGTTACGGGGCACTAATTTCCCTAGTGACGGGCCAGT
TATGCAAAAGAAGACGATGGGATGGGAAGCCAGTTCAGAGAGGATGTACC
CAGAGGACGGTGCCTTAAAGGGAGAGATCAAGCAACGACTCAAGTTGAAG
GACGGTGGACACTACGACGCTGAGGTAAAGACGACGTACAAGGCCAAGAA
GCCAGTACAACTCCCCGGAGCTTTTGTCGA

In certain embodiment(s), SEQ ID 22 (as submitted herewith in the Sequence Listing) is of length 1952, of molecular type of DNA, of synthetic construct, is referred to “SBP134,” and is of the following sequence structure.

GAATTCCAGCTGTAATACGACTCACTATAGGAGGGAAAAGTTATCAGGCA
TGCACCTGGTAGCTAGTCTTTAAACCAATAGATTGCATCGGTTTAAAAGG
CAAGACCGTCAAATTGCGGGAAAGGGGTCAACAGCCGTTCAGTACCAAGT
CTCAGGGGAAACTTTGAGATGGCCTTGCAAAGGGTATGGTAATAAGCTGA
CGGACATGGTCCTAACCACGCAGCCAAGTCCTAAGTCAACAGATCTTCTG
TTGATATGGATGCAGTTCACAGACTAAATGTCGGTCGGGGAAGATGTATT
CTTCTCATAAGATATAGTCGGACCTCTCCTTAATGGGAGCTAGCGGATGA
AGTGATGCAACACTGGAGCCGCTGGGAACTAATTTGTATGCGAAAGTATA
TTGATTAGTTTTGGAGTACTCGTCCATTCGCATGGGACATATTAAGTCCT
CAATTCATGTACGGGTCTAAGGCATACGTTAAGCACCCCGCAGACATACC
AGACTACTTGAAGCTCTCCTTCCCCGAGGGATTCAAGTGGGAGAGGGTCA
TGAATTTCGAGGACGGTGGGGTAGTTACGGTTACTCAAGACTCTTCCTTA
CAAGACGGAGAGTTCATATACAAGGTTAAGTTACGGGGCACTAATTTCCC
TAGTGACGGGCCAGTTATGCAAAAGAAGACGATGGGATGGGAAGCCAGTT
CAGAGAGGATGTACCCAGAGGACGGTGCCTTAAAGGGAGAGATCAAGCAA
CGACTCAAGTTGAAGGACGGTGGACACTACGACGCTGAGGTAAAGACGAC
GTACAAGGCCAAGAAGCCAGTACAACTCCCCGGAGCTTACAATGTTAATA
TCAAGCTTGACATCACTTCCCACAATGAGGACTACACAATCGTTGAGCAA
TACGAGCGGGCCGAGGGACGGCACTCAACAGGTGGAATGGACGAGCTCTA
CAAGTAGAAAAACAAAAACAAAAACAAAAACAAAAACAAAAACAAAAACA
AAAACCAAAAACAAAAACAAAAACACACATTAAAACAGCCTGTGGGTTGA
TCCCACCCACAGGCCCATTGGGCGCTAGCACTCTGGTATCACGGTACCTT
TGTGCGCCTGTTTTATACCCCCTCCCCCAACTGTAACTTAGAAGTAACAC
ACACCGATCAACAGTCAGCGTGGCACACCAGCCACGTTTTGATCAAGCAC
TTCTGTTACCCCGGACTGAGTATCAATAGACTGCTCACGCGGTTGAAGGA
GAAAGCGTTCGTTATCCGGCCAACTACTTCGAAAAACCTAGTAACACCGT
GGAAGTTGCAGAGTGTTTCGCTCAGCACTACCCCAGTGTAGATCAGGTCG
ATGAGTCACCGCATTCCCCACGGGCGACCGTGGCGGTGGCTGCGTTGGCG
GCCTGCCCATGGGGAAACCCATGGGACGCTCTAATACAGACATGGTGCGA
AGAGTCTATTGAGCTAGTTGGTAGTCCTCCGGCCCCTGAATGCGGCTAAT
CCTAACTGCGGAGCACACACCCTCAAGCCAGAGGGCAGTGTGTCGTAACG
GGCAACTCTGCAGCGGAACCGACTACTTTGGGTGTCCGTGTTTCATTTTA
TTCCTATACTGGCTGCTTATGGTGACAATTGAGAGATCGTTACCATATAG
CTATTGGATTGGCCATCCGGTGACTAATAGAGCTATTATATATCCCTTTG
TTGGGTTTATACCACTTAGCTTGAAAGAGGTTAAAACATTACAATTCATT
GTTAAGTTGAATACAGCAAAATGGTCAGCAAGGGCGAGGAAGACAATATG
GCAATCATCAAGGAGTTCATGAGATTCAAGGTTCACATGGAAGGTTCTGT
CAATGGGCACGAGTTCGAGATCGAGGGAGAGGGAGAGGGAAGGCCATACG
AGGGAACACAAACAGCCAAGTTAAAGGTCACGAAGGGCGGCCCTCTGTCG
AC

In certain embodiment(s), SEQ ID 23 (as submitted herewith in the Sequence Listing) is of length 1810, of molecular type of DNA, of synthetic construct, is referred to “SBP138,” and is of the following sequence structure.

GAATTCCAGCTGTAATACGACTCACTATAggaaGAGCCTTTATACAGTAA
TGTATATCGAAAAATCCTCTAATTCAGGGAACACCTAAACAAACTAAGAT
GTAGGCAATCCTGAGCTAAGCTCTTAGTAATAAGAGAAAGTGCAACGACT
ATTCCGATAGGAAGTAGGGTCAAGTGACTCGAAATGGGGATTACCCTTCT
AGGGTAGTGATATAGTCTGAACATATATGGAAACATATAGAAGGATAGGA
GTAACGAACCTATCCGTAACATAATTGACAATGTTAATATCAAGCTTGAC
ATCACTTCCCACAATGAGGACTACACAATCGTTGAGCAATACGAGCGGGC
CGAGGGACGGCACTCAACAGGTGGAATGGACGAGCTCTACAAGTAGAAAA
ACAAAAACAAAAACAAAAACaAAAAACAAAAACAAAAACAAAAACCAAAA
ACAAAAACAAAAACACACATTAAAACAGCCTGTGGGTTGATCCCACCCAC
CCTGTTTTATACCCCCTCCCCCAACTGTAACTTAGAAGTAACACACACCG
AGGCCCATTGGGCGCTAGCACTCTGGTATCACGGTACCTTTGTGCGATCA
CTGAGTATCAATAGACTGCTCACGCGGTTGAAGGAGAAAGCGTTCGTTAT
ACAGTCAGCGTGGCACACCAGCCACGTTTTGATCAAGCACTTCTGTTACC
CCGGACCGGCCAACTACTTCGAAAAACCTAGTAACACCGTGGAAGTTGCA
GAGTGTTTCGCTCAGCACTACCCCAGTGTAGATCAGGTCGATGAGTCACC
GCATTCCCCACGGGCGACCGTGGCGGTGGCTGCGTTGGCGGCCTGCCCAT
GGGGAAACCCATGGGACGCTCTAATACAGACATGGTGCGAAGAGTCTATT
GAGCTAGTTGGTAGTCCTCCGGCCCCTGAATGCGGCTAATCCTAACTGCG
GAGCACACACCCTCAAGCCAGAGGGCAGTGTGTCGTAACGGGCAACTCTG
CAGCGGAACCGACTACTTTGGGTGTCCGTGTTTCATTTTATTCCTATACT
GGCTGCTTATGGTGACAATTGAGAGATCGTTACCATATAGCTATTGGATT
GGCCATCCGGTGACTAATAGAGCTATTATATATCCCTTTGTTGGGTTTAT
ACCACTTAGCTTGAAAGAGGTTAAAACATTACAATTCATTGTTAAGTTGA
ATACAGCAAAATGGTCAGCAAGGGCGAGGAAGACAATATGGCAATCATCA
AGGAGTTCATGAGATTCAAGGTTCACATGGAAGGTTCTGTCAATGGGCAC
GAGTTCGAGATCGAGGGAGAGGGAGAGGGAAGGCCATACGAGGGAACACA
AACAGCCAAGTTAAAGGTCACGAAGGGCGGGCCTCTTCCATTCGCATGGG
ACATATTAAGTCCTCAATTCATGTACGGGTCTAAGGCATACGTTAAGCAC
CCCGCAGACATACCAGACTACTTGAAGCTCTCCTTCCCCGAGGGATTCAA
GTGGGAGAGGGTCATGAATTTCGAGGACGGTGGGGTAGTTACGGTTACTC
AAGACTCTTCCTTACAAGACGGAGAGTTCATATACAAGGTTAAGTTACGG
GGCACTAATTTCCCTAGTGACGGGCCAGTTATGCAAAAGAAGACGATGGG
ATGGGAAGCCAGTTCAGAGAGGATGTACCCAGAGGACGGTGCCTTAAAGG
GAGAGATCAAGCAACGACTCAAGTTGAAGGACGGTGGACACTACGACGCT
GAGGTAAAGACGACGTACAAGGCCAAGAAGCCAGTACAACTCCCCGGAGC
TTttGTCGAC

In certain embodiment(s), the term “isolated” refers to that a sequence or a material is present in an environment separate and distinct from a natural organism or entity. In certain embodiment(s), the term “isolated” refers to a laboratory procedure where a sequence is synthesized or modified using a chemical process.

In certain embodiment(s), the nucleic acid sequence is with at least 90% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21, wherein the at least 90% similarity refers to that at least 9 nucleotides are the same for every 10 nucleotides between two sequences, in a 5′ to 3′ direction.

In certain embodiment(s), the nucleic acid sequence is SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21.

In certain embodiment(s), the nucleic acid sequence is with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09 or SEQ ID NO. 20.

In certain embodiment(s), the nucleic acid sequence is with at least 90% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09 or SEQ ID NO. 20.

In certain embodiment(s), the nucleic acid sequence is SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09 or SEQ ID NO. 20.

In certain embodiment(s), the nucleic acid sequence is with at least 80% similarity to SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09 or SEQ ID NO. 20.

In certain embodiment(s), the nucleic acid sequence is with at least 90% similarity to SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09 or SEQ ID NO. 20.

In certain embodiment(s), the nucleic acid sequence is SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09 or SEQ ID NO. 20.

In certain embodiment(s), the nucleic acid sequence is with at least 80% similarity to SEQ ID NO. 12, or SEQ ID NO. 21.

In certain embodiment(s), the nucleic acid sequence is with at least 90% similarity to SEQ ID NO. 12, or SEQ ID NO. 21.

In certain embodiment(s), the nucleic acid sequence is SEQ ID NO. 12, or SEQ ID NO. 21.

In a second aspect, the present disclosure provides a DNA (deoxyribonucleic acid) construct. In certain embodiment(s), the DNA construct includes a ribozyme sequence, where an RNA segment transcribed from the ribozyme sequence includes an isolated nucleic acid sequence referenced in the first aspect, for example, the isolated nucleic acid is with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21, wherein the at least 80% similarity refers to that at least 8 nucleotides are the same for every 10 nucleotides between two sequences, in a 5′ to 3′ direction.

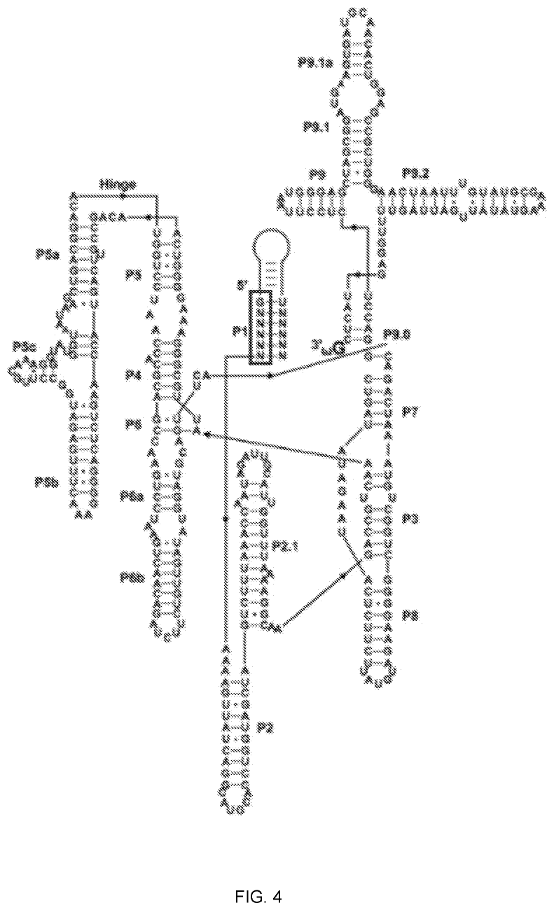

In certain embodiment(s), the RNA segment transcribed from the ribozyme sequence is segment S1, and the DNA construct further includes a target sequence, where an RNA segment transcribed from the target sequence is segment S2, and wherein IGS of segment S1 is complementary in base pair to the segment S2. With further reference to FIG. 3, the segment named “Ribozyme” is a non-limiting example of segment S1, and the segment named “CCCUCU” is a non-limiting example of segment S2. As shown in FIG. 3, numeral “1” represents the DNA construct, numeral “2” represents the RNA transcript. Arrow “3a” and arrow “3b” represent the position of the nucleophilic attack, numeral “4” represents the circular IRES+GOI, the star sign “*” represents the G:U wobble base pair.

In certain embodiment(s), and in view of FIG. 3, a wobble base pair G-U is present between the segment S1 and the segment S2.

In certain embodiment(s), the DNA construct further includes a sequence of interest, positioned between the ribozyme sequence and the target sequence. With further reference to FIG. 3, the segment named “IRES+GOI” is a non-limiting example of the sequence of interest.

In certain embodiment(s), the segment S1 includes a sequence of GGAGGG.

In certain embodiment(s), the segment S1 includes a sequence of GGAGGG, and the sequence of GGAGGG is in a 5′ to 3′ direction.

In certain embodiment(s), wherein the segment S2 includes a sequence of CCCUCU.

In certain embodiment(s), the DNA construct further includes an IGS (internal guide sequence) which is upstream part of the ribozyme sequence.

In certain embodiment(s), the term “internal guide sequence” (IGS) refers to a specific nucleotide sequence within a ribozyme or added to the ribozyme sequence that is responsible for recognizing and base pairing with a complementary target sequence.

In certain embodiment(s), the IGS includes a 5′-GN₁ . . . Nₙ-3′ sequence, wherein N represents any one of adenine (A), guanine (G), cytosine (C), and uracil (U), and where n represents a positive integer, greater than 2 smaller than 10.

In certain embodiment(s), n is 5, and the IGS includes a 5′-GNNNNN-3′ sequence. The guanine (G) at the 5′ end of the IGS forms a G-U wobble pair with the uracil (U) at the 3′ end of the target sequence. This base pairing interaction guides the ribozyme to the correct splice site and induces an activation of the catalytic activity of the ribozyme. The IGS serves as an internal guide or template for the circularization process, promoting the formation of a covalently closed circular RNA structure from the linear precursor.

Examples of IGS used in RNA circularization methods include complementary sequences or stem-loop structures or RNA binding protein recognition sequences. The design and selection of the IGS are particular in achieving efficient RNA circularization. The IGS sequence or structure are designed with an aim for proper folding, stability, and compatibility with the circularization mechanism being employed.

In certain embodiment(s), the IGS (Internal guide sequence) is designed such that the first nucleotide of the IGS is G followed by GNNNN . . . N, where N is any nucleotide, number of Ns is 3 to 7. In this design, target sequence is reverse complimentary to the IGS such that a GU wobble base pairing is formed at the end of the sequence.

In certain embodiment(s), the ribozyme sequence is of viral origin, of fungal origin, or of bacterial origin.

In a third aspect, the present disclosure provides a method of forming circular RNA. In certain embodiment(s), the method includes providing a DNA (deoxyribonucleic acid) construct referenced in the second aspect, in particular, the DNA constructs is provided to include a ribozyme sequence, where an RNA segment transcribed from the ribozyme sequence includes a nucleic acid sequence with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21, wherein the at least 80% similarity refers to that at least 8 nucleotides are the same for every 10 nucleotides between two sequences, in a 5′ to 3′ direction.

In certain embodiment(s), the present disclosure provides a method of making circularization of an RNA of interest without a use of permuted intron-exon (PIE), the method including: providing a DNA (deoxyribonucleic acid) construct, wherein the DNA construct includes a Tetrahymena ribozyme or Twort ribozyme or one or more of their respective variants, a sequence of interest, and a target sequence; causing an RNA to be transcribed from the DNA construct, the RNA includes a segment S1 corresponding to the Tetrahymena ribozyme, a segment S2 corresponding to the target sequence, and a segment S3 corresponding to the sequence of interest; and effecting a base-pairing between the segment S1 and the segment S2, wherein the base-pairing includes circularization of the segment S3.

In certain embodiment(s), the term “intron-exon permutation” (PIE) refers to a rearrangement of natural order of introns and exons in a gene, which may be used to engineer ribozymes for trans-splicing. In nature, group I introns are typically found in a specific order relative to their flanking exons. However, this order may be permuted to create engineered ribozymes that are capable of trans-splicing. The present disclosure in certain embodiment(s) is unique and advantageous at least in not requiring intron-exon permutation to achieve circularization of an RNA sequence.

In certain embodiment(s), the term “scarless circularization” refers to a process of excising an RNA sequence and joining its ends to form a circular RNA molecule without introducing any nucleotides from the ribozyme or other intron. In certain particular embodiment(s), the group I intron ribozyme recognizes the target sequence via the IGS, catalyzes the excision of the target sequence, and ligates the ends of the target sequence to form a circular RNA. This process is considered “scarless” because the resulting circular RNA contains the nucleotides of the sequence of interest and does not contain any nucleotides from the ribozyme or other introns.

In a fourth aspect, the present disclosure provides an isolated sequence primer for detecting the presence of circular RNA. In certain embodiment(s), the isolated sequence primer includes a nucleic acid sequence with at least 80% (80 percent) similarity to: a nucleic acid sequence of AAGGACGGTGGACACTACGA, in a 5′ to 3′ direction; a nucleic acid sequence of CTTTCTCCTTCAACCGCGTG, in a 5′ to 3′ direction; a nucleic acid sequence of GGAAGGTTCTGTCAATGGGC, in a 5′ to 3′ direction; nucleic acid sequence of TCTTGGCCTTGTACGTCGTC, in a 5′ to 3′ direction; a nucleic acid sequence of GACGACGTACAAGGCCAAGA, in a 5′ to 3′ direction; or a nucleic acid sequence of AAGTTACAGTTGGGGGAGGG, in a 5′ to 3′ direction, where the at least 80% similarity refers to that at least 8 nucleotides are the same for every 10 nucleotides between two sequences, in a 5′ to 3′ direction.

In certain embodiment(s), the nucleic acid sequence of the primer is with at least 90% similarity to: the nucleic acid sequence of AAGGACGGTGGACACTACGA, in the 5′ to 3′ direction; the nucleic acid sequence of CTTTCTCCTTCAACCGCGTG, in the 5′ to 3′ direction; the nucleic acid sequence of GGAAGGTTCTGTCAATGGGC, in the 5′ to 3′ direction; the nucleic acid sequence of TCTTGGCCTTGTACGTCGTC, in the 5′ to 3′ direction; the nucleic acid sequence of GACGACGTACAAGGCCAAGA, in the 5′ to 3′ direction; or the nucleic acid sequence of AAGTTACAGTTGGGGGAGGG, in the 5′ to 3′ direction, where the at least 90% similarity refers to that at least 9 nucleotides are the same for every 10 nucleotides between two sequences, in a 5′ to 3′ direction.

In certain embodiment(s), the nucleic acid sequence of the primer is: the nucleic acid sequence of AAGGACGGTGGACACTACGA, in the 5′ to 3′ direction; the nucleic acid sequence of CTTTCTCCTTCAACCGCGTG, in the 5′ to 3′ direction; the nucleic acid sequence of GGAAGGTTCTGTCAATGGGC, in the 5′ to 3′ direction; the nucleic acid sequence of TCTTGGCCTTGTACGTCGTC, in the 5′ to 3′ direction; the nucleic acid sequence of GACGACGTACAAGGCCAAGA, in the 5′ to 3′ direction; or the nucleic acid sequence of AAGTTACAGTTGGGGGAGGG, in the 5′ to 3′ direction.

In certain embodiment(s), the term “target sequence” refers to an RNA sequence that is recognized by the ribozyme's IGS to form a G-U wobble base pair. In certain particular embodiment(s), the target sequence includes or is a 5′-N′N′N′N′N′U-3′ sequence, where N′ represents any nucleotide, and the uracil (U) at the 3′ end forms a G-U wobble pair with the guanine (G) at the 5′ end of the ribozyme's IGS. The target sequence may be any RNA sequence, such as a coding sequence, untranslated region, or regulatory element.

In certain embodiment(s), the term “trans-esterification” refers to a chemical reaction mechanism by which the ribozyme catalyzes the cleavage and ligation of RNA molecules during the splicing process. In a first step of trans-esterification, the 3′-hydroxyl group of an exogenous guanosine nucleotide attacks the phosphodiester bond at the 5′ splice site, cleaving the RNA and forming a 3-hydroxyl group on the 5′ exon and a 3′-guanosine monophosphate on the intron. In a second step of trans-esterification, the 3-hydroxyl group of the 5′ exon attacks the phosphodiester bond at the 3′ splice site, ligating the exons and releasing the intron. The present disclosure in certain embodiment(s) harnesses trans-esterification mechanism to catalyze the excision and circularization of an RNA sequence.

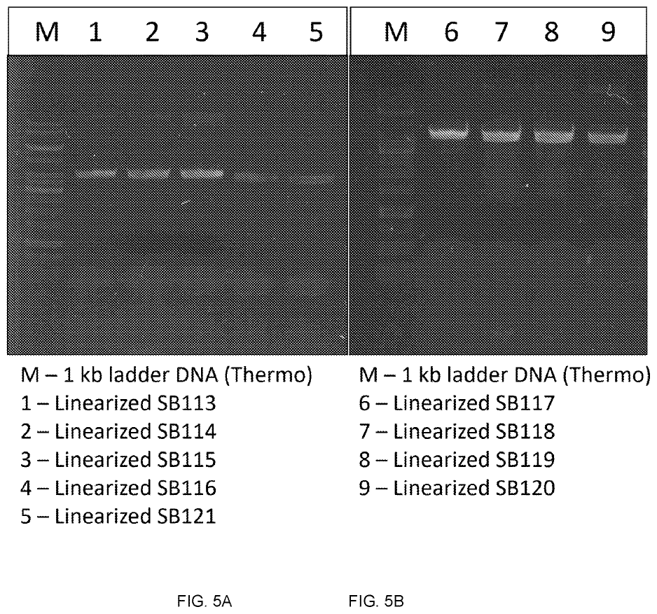

In certain embodiment(s), the term “group I intron” refers to a class of self-splicing ribozymes found in various organisms, including bacteria, fungi, virus, and algae. Group I introns are characterized by their distinct secondary and tertiary structures, which are particular for their catalytic activity. These introns catalyze their own excision from precursor RNA molecules through a two-step trans-esterification reaction, as described herein. The core structure of group I introns consists of conserved paired elements (P1-P10), as illustratively shown in FIG. 4 and conserved sequences, such as the internal guide sequence (IGS) and the guanosine binding site. The present disclosure in certain embodiment(s) utilizes engineered group I introns to catalyze the scarless circularization of an RNA sequence.

In certain embodiment(s), the term “cis-splicing” and the term “trans-splicing” refer to RNA molecule splicing. Cis-splicing occurs when the intron catalyzes its own excision from a contiguous RNA molecule, meaning that the intron is located within the same RNA molecule as its flanking exons. In contrast, trans-splicing involves the ribozyme acting on a separate target RNA molecule. In trans-splicing, the ribozyme and the RNA sequence of interest are two different and separate RNA molecules.

In certain embodiment(s), the term “circular RNA” or “circRNA” refers to a single-stranded RNA molecule in which the 3′ and 5′ ends are covalently linked to form a continuous loop structure, without any free ends. Circular RNAs are naturally occurring molecules that have been found in various organisms, including humans. They are generated through a process called back-splicing, in which a downstream 5′ splice site is ligated to an upstream 3′ splice site. Circular RNAs have been shown to have various biological functions, such as acting as microRNA sponges, regulating gene expression, and encoding proteins. The present disclosure in certain embodiment(s) enables an efficient and scarless production of circular RNAs containing any desired sequence of interest.

In certain embodiment(s), the term “RNA” or “RNA molecule” or “RNA structure” refers to a three-dimensional conformation of an RNA molecule, determined by its nucleotide sequence and stabilized by various interactions, such as base pairing, base stacking, and hydrogen bonding. RNA molecules may form diverse secondary structures, such as hairpins, loops, bulges, and pseudoknots, which are essential for their biological functions.

In certain embodiment(s), the term “gene” refers to a segment of deoxyribonucleic acid (DNA) or ribonucleic acid (RNA) that encodes a particular function. In the context of RNA technology, genes are often involved in the transcription process, where specific genes are transcribed from DNA into RNA molecules, which may then be translated into proteins or serve other functional roles.

In certain embodiment(s), the term “convergent primer” and the term “divergent primer” refer to primers that are short, synthetic oligonucleotide sequences which serve as starting points for DNA synthesis or amplification reactions. Convergent primers are a pair of primers designed to amplify a particular region of a nucleic acid sequence, such as a gene or a DNA/RNA fragment. Convergent primers are oriented in opposite directions (5′ to 3′ and 3′ to 5′) and bind to the complementary strands of a template sequence, allowing the amplification process to proceed inwardly towards each other. Divergent primers, on the other hand, are a pair of primers designed to amplify a region in an outward direction, starting from a common sequence and moving away from each other. Divergent primers are oriented in the same direction (both 5′ to 3′ or both 3′ to 5′) and bind to the same strand of the template sequence.

In certain embodiment(s), the term “promoter” refers to a specific DNA sequence located upstream (towards the 5′ end) of a gene that serves as a binding site for transcription factors and RNA polymerase enzymes. The promoter plays an important role in initiating and regulating the transcription of a gene into RNA molecules. Promoters are important for controlling the expression of genes involved in various RNA processes, such as RNA interference (RNAi), RNA editing, or the production of functional non-coding RNAs. In the context of RNA circularization methods, a promoter refers to a specific DNA sequence that is used to drive the transcription of linear RNA molecules, which may subsequently be circularized or cyclized into circular RNA (circRNA) structures. Promoters play an important role in initiating and controlling the transcription of the linear RNA precursor that is subsequently circularized. Examples of promoters that are suitable for RNA circularization include T7 promoter, U6 promoter or CMV promoter.

In certain embodiment(s), T7 promoter is employed. The T7 promoter is a viral promoter derived from the T7 bacteriophage. T7 promoter is particular for in vitro transcription of RNA molecules, including circular RNAs. The T7 promoter is specific and recognized by the T7 RNA polymerase, allowing for transcription of linear RNA precursors.

In certain embodiment(s), U6 promoter is employed. The U6 promoter is a RNA polymerase III promoter found in the U6 small nuclear RNA (snRNA) gene. U6 promoter is particular for the expression of short hairpin RNAs (shRNAs) and small interfering RNAs (siRNAs) in RNA interference (RNAi) studies. U6 promoter may also be employed for the transcription of linear RNA precursors that may be circularized.

In certain embodiment(s), cytomegalovirus (CMV) promoter is employed. The CMV promoter is a promoter derived from the human cytomegalovirus. The CMV is used for transgene expression in mammalian cells and may be employed for the transcription of linear RNA precursors destined for circularization.

Any of suitable promoters may be employed, in consideration for efficiency of transcription and the subsequent circularization process, as well as the cellular context in which the circular RNAs are produced, whether in vivo or in vitro.

In certain embodiment(s), the term “in vitro transcription” (IVT) refers to a technique used to produce linear RNA molecules from a DNA template. These linear RNA molecules may then be subsequently circularized or cyclized into circular RNA (circRNA) structures. The IVT method for RNA circularization, include a) DNA template preparation, b) in vitro transcription reaction, c) purification and d) circularization. A DNA template containing the desired sequence for the linear RNA precursor is prepared. This template includes the promoter sequence recognized by the RNA polymerase enzyme and the coding sequence for the linear RNA molecule. During In vitro transcription reaction, the DNA template is incubated with the appropriate RNA polymerase enzyme (for example, T7 RNA polymerase, SP6 RNA polymerase), ribonucleotide triphosphates (NTPs), and other necessary reaction components (for example, buffer, divalent cations) in a cell-free environment. The RNA polymerase recognizes the promoter sequence and transcribes the linear RNA molecule from the DNA template. Subsequently, the linear RNA is purified. The transcribed linear RNA molecules are typically purified from the reaction mixture using various techniques, such as gel electrophoresis, chromatography, or precipitation methods, to remove the DNA template, enzymes, and other reaction components. The purified linear RNA molecules are subjected to specific circularization methods, such as RNA ligation, splicing, or complementary sequence-based cyclization. These methods facilitate the formation of a covalently closed circular RNA structure from the linear precursor.

In certain embodiment(s), the present disclosure provides a method for intramolecular circularization of RNA using group 1 introns like Tetrahymena ribozyme or Twort ribozyme or one or more of their respective variants without the use of permuted intron-exon. Synthetic variants of Tetrahymena ribozyme or Twort ribozyme are designed and employed to enhance circularization efficiency. The circularization method produces scarless circRNAs.

In yet another aspect, the present disclosure provides a method of making circRNA that utilizes a prototype plasmid into which sequence of interest for the desired circRNA is inserted. In certain particular embodiment(s), the plasmid construct includes, sequence downstream to T7 promoter, a) internal guide region (IGS), b) ribozyme sequence, c) sequence of interest, d) target. In certain particular embodiment(s), the ribozyme sequence includes wild type sequence or its sequence variant.

In certain embodiment(s), a portion, a whole, or a variant of SEQ. ID. NO. 1. encodes a wild-type Tetrahymena ribozyme.

In certain embodiment(s), a portion, a whole, or a variant of SEQ. ID. NO. 2. encodes an engineered Tetrahymena ribozyme.

In certain embodiment(s), a portion, a whole, or a variant of SEQ. ID. NO. 3. encodes an engineered Tetrahymena ribozyme.

In certain embodiment(s), a portion, a whole, or a variant of SEQ. ID. NO. 4. encodes an engineered Tetrahymena ribozyme.

In certain embodiment(s), a portion, a whole, or a variant of SEQ. ID. NO. 5. encodes an engineered Tetrahymena ribozyme.

In certain embodiment(s), a portion, a whole, or a variant of SEQ. ID. NO. 6. encodes an engineered Tetrahymena ribozyme.

In certain embodiment(s), a portion, a whole, or a variant of SEQ. ID. NO. 7. encodes an engineered Tetrahymena ribozyme.

In certain embodiment(s), a portion, a whole, or a variant of SEQ. ID. NO. 8. encodes an engineered Tetrahymena ribozyme.

In certain embodiment(s), a portion, a whole, or a variant of SEQ. ID. NO. 9. encodes an engineered Tetrahymena ribozyme.

In certain embodiment(s), a portion, a whole, or a variant of SEQ. ID. NO. 10. encodes an Anabaena ribozyme.

In certain embodiment(s), a portion, a whole, or a variant of SEQ. ID. NO. 11. encodes an Azoarcus ribozyme.

In certain embodiment(s), a portion, a whole, or a variant of SEQ. ID. NO. 12. encodes a Twort ribozyme.

In certain embodiment(s), a portion, a whole, or a variant of SEQ. ID. NO. 13. encodes a Scytalidium ribozyme.

In certain embodiment(s), a portion, a whole, or a variant of SEQ. ID. NO. 14. encodes a Clostridium ribozyme.

In certain embodiment(s), a portion, a whole, or a variant of SEQ. ID. NO. 20, encodes an engineered Tetrahymena ribozyme.

In certain embodiment(s), a portion, a whole, or a variant of SEQ. ID. NO. 21, encodes an engineered Twort ribozyme.

In certain embodiment(s), in vitro transcription using T7 polymerase (SEQ. ID. NO. 15) generates RNA with base complementarity between the IGS and the target sequence, where the base complementarity brings the ends of the transcripts together. In the presence of GTP, the activated ribozyme is spliced out via two transesterification reactions such that the downstream components to the ribozymes including the target sequence are circularized, as illustratively shown in FIG. 3.

The present invention is described more in detail below in the following Examples, but the scope of the present invention is not limited thereto. The following Examples are non-limiting and other variants contemplated by one of ordinary skill in the art are included within the scope of this disclosure.

### EXAMPLES

### Example 1: Method of Intramolecular RNA Circularization

In Example 1, the method involves the circularization of linear RNA using group 1 introns like the Tetrahymena ribozyme-enabled intra-molecular splicing process. The design does not include the permutation of intron and exon sequences.

The DNA construct includes an internal guide region (IGS), a ribozyme sequence (WT or Variants of Tetrahymena or Twort), a sequence of interest, and a target sequence.

The DNA constructs include the mentioned components downstream to a T7 promoter. In vitro transcription using T7 polymerase generates RNA with base complementarity between the IGS and the target sequence bringing the ends of the transcripts together. In the presence of GTP, the activated ribozyme is spliced out via two trans-esterification reactions such that the downstream components to the ribozyme including the target sequence are circularized, as illustratively shown in FIG. 3.

### Example 2: Construct Design Using Natural Tetrahymena Ribozyme or Twort and their Respective Variants

Eight Tetrahymena ribozyme-based circular RNA generating constructs are designed, namely SBP114 to 121 in additional to the WT (wild-type), namely SBP113. The designed circular RNA generating constructs are cloned into appropriate vector systems downstream of T7 promoter. The term “SBP” refers to a nomenclature of a plasmid.

Following plasmids are synthesized.

Plasmid SBP113 (SEQ. ID. NO. 17) is a WT (wild-type) Tetrahymena ribozyme-based circular RNA construct, encoding mCherry (SEQ. ID. NO. 16), where mCherry is a non-limiting example of a sequence of interest.

Plasmid SBP 114 to SBP 121 are each a variant to SBP113.

Plasmid SBP114 is a circular RNA construct which includes a variant (double mutation) of WT Tetrahymena ribozyme, encoding mCherry.

Plasmid SBP115 is a circular RNA construct which includes a variant of WT Tetrahymena ribozyme, encoding mCherry.

Plasmid SBP116 is a circular RNA construct which includes a variant (mutation in P9.2 domain) of WT Tetrahymena ribozyme, encoding mCherry.

Plasmid SBP117 is a circular RNAconstruct which includes a variant of (double mutation) of a WT Tetrahymena ribozyme, encoding mCherry.

Plasmid SBP118 is a circular RNA construct which includes a variant (P1extendedP10 loop forming) of WT Tetrahymena ribozyme.

Plasmid SBP119 is a circular RNA construct which includes a variant (double mutation) of WT Tetrahymena ribozyme, encoding mCherry.

Plasmid SBP120 is a circular RNA construct which includes a variant (P1extendedP10 loop forming double mutation) of WT Tetrahymena ribozyme, encoding mCherry.

Plasmid SBP121 is a circular RNA construct which includes a variant (splicing inefficient) of WT Tetrahymena ribozyme, encoding mCherry.

### Example 3: DNA Propagation and Linearization

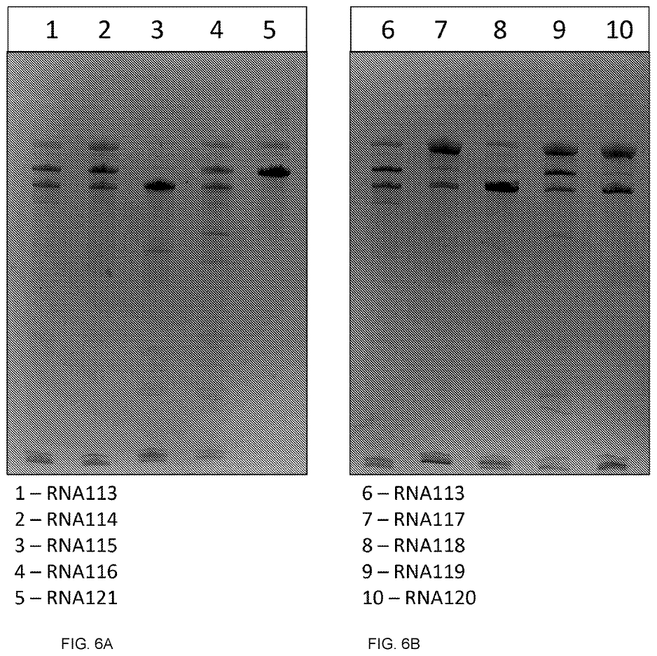

All the synthesized plasmids are propagated in Escherichia coli and isolated and purified using plasmid isolation kits following the manufacturer's protocols. The purified plasmids are linearized and the products are visualized by agarose gel electrophoresis, as illustratively shown in FIG. 5A and FIG. 5B. As shown in FIG. 5A and FIG. 5B, lane “M” represents a 1 kb ladder DNA, lane “1 represents a linearized SB113, lane “2” represents a linearized SB114, lane 3” represents linearized SB115, lane “4” represents linearized SB116, lane “5 represents linearized SB121, lane “6” represents linearized SB117, lane 7” represents linearized SB11, lane “8” represents linearized SB119, and lane “9” represents linearized SB120.

All the designed plasmids are isolated from E. coli and linearized before in vitro transcription is performed. After overnight incubation at 37° C. with the appropriate restriction enzyme, linearization of the plasmid is observed.

### Example 4: In Vitro Transcription of all Constructs—Single Step IVT and Circularization of RNA

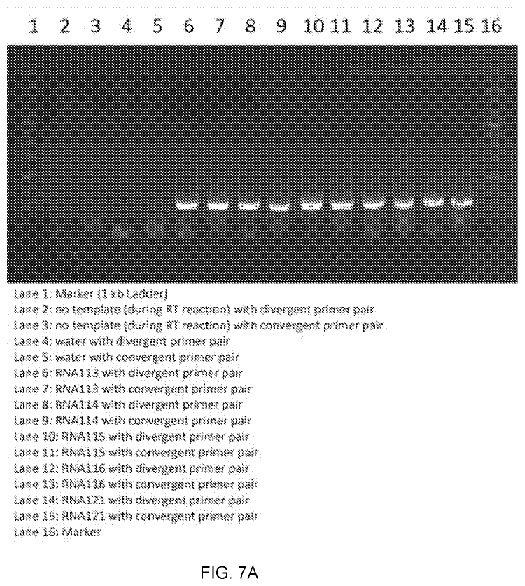

Linearized plasmid DNA (SBP113 to SBP121) is used in an in vitro transcription reaction to synthesize RNA (RNA113 to RNA121) without added circularization manipulation. As illustratively shown in FIG. 6A and FIG. 6B, multiple bands were observed when the RNAs are subjected to Urea-PAGE analyses. As shown in FIG. 6A and FIG. 6B, lane “1” represents RNA113, lane “2” represents RNA114, lane “3” represents RNA115, lane “4” represents RNA116, lane “5” presents RNA121, lane “6” represents RNA113, lane “7” represents RNA117, lane “8” represents RNA118, lane “9” represents RNA120.

RNA is purified using the Monarch nucleic acid clean-up kit, and 1 ug of each purified RNA is subjected to 4% Urea-PAGE analysis. Bands on the gel are visualized via staining with toluidine blue.

### Example 5: Detection of Circular RNA

Presence of circular RNA may be detected using RTPCT. RTPCR with convergent and divergent primer (a qualitative assay for detection of circular RNA) is used to detect the presence of circular RNA. The RT PCR is performed using two sets of primer pairs. The first pair, or the convergent primer pair, results in a PCR product from both linear and circular RNA post cDNA synthesis. The second pair, or the divergent primer pair, results in a PCR product only when cDNA is generated from circular RNA.

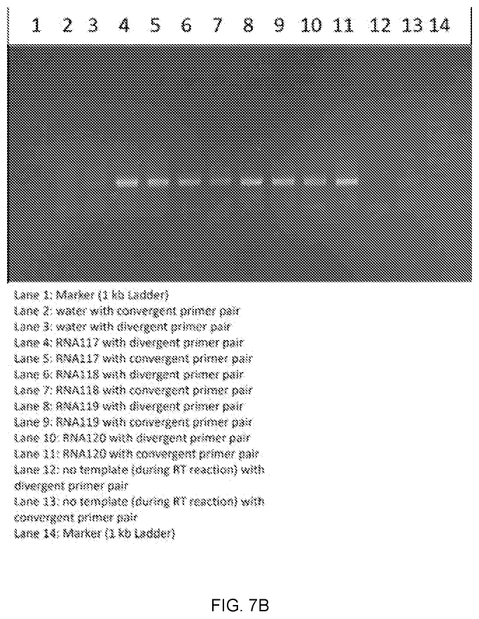

After cDNA synthesis using the iScript cDNA synthesis kit (Biorad), PCR products with both convergent and divergent primer pairs are observed for all the generated RNA transcripts, indicating the presence of circular RNA, as illustratively shown in FIG. 7A and FIG. 7B. 1 μg of each RNA was used for the generation cDNA using the manufacturer's protocol before performing the PCR reactions.

FIG. 7A and FIG. 7B are each a schematic visualization of RT-PCR products obtained from convergent and divergent primer pairs on a 0.8% agarose gel, where PCR products at the expected size are observed, indicating circular RNA presence in all the samples tested.

As shown in FIG. 7A, lane 1 corresponds to a 1 kb ladder marker, lane 2 corresponds to no template (during RT reaction) with divergent primer pair, lane 3 corresponds to no template (during RT reaction) with convergent primer pair, lane 4 corresponds to water with divergent primer pair, lane 5 corresponds to water with convergent primer pair, lane 6 corresponds to RNA113 with divergent primer pair, lane 7 corresponds to RNA113 with convergent primer pair, lane 8 corresponds to RNA114 with divergent primer pair, lane 9 corresponds to RNA114 with convergent primer pair, lane 10 corresponds to RNA115 with divergent primer pair, lane 11 corresponds to RNA115 with convergent primer pair, lane 12 corresponds to RNA116 with divergent primer pair, lane 13 corresponds to RNA116 with convergent primer pair, lane 14 corresponds to RNA121 with divergent primer pair, lane 15 corresponds to RNA121 with convergent primer pair, and lane 16 corresponds to marker.

As shown in FIG. 7B, lane 1 corresponds to a 1 kb ladder marker, lane 2 corresponds to water with convergent primer pair, lane 3 corresponds to water with divergent primer pair, lane 4 corresponds to RNA117 with divergent primer pair, lane 5 corresponds to RNA 117 with convergent primer pair, lane 6 corresponds to RNA118 with divergent primer pair, lane 7 corresponds to RNA118 with convergent primer pair, lane 8 corresponds to RNA119 with divergent primer pair, lane 9 corresponds to RNA119 with convergent primer pair, lane 10 corresponds to RNA120 with divergent primer pair, lane 11 corresponds to RNA120 with convergent primer pair, lane 12 corresponds to no template (during RT reaction) with divergent primer pair, lane 13 corresponds to no template (during RT reaction) with convergent primer pair, and lane 14 corresponds to 1 kb ladder marker.

Alternatively, presence of circular RNA may be detected using RNase R treatment and urea page. To confirm the presence of circular RNA, the generated RNA transcripts are treated with RNase R. RNase R is an exonuclease that specifically cleaves linear RNA and does not degrade circular RNA. Hence, this enzyme is used for the identification and enrichment of circular RNA.

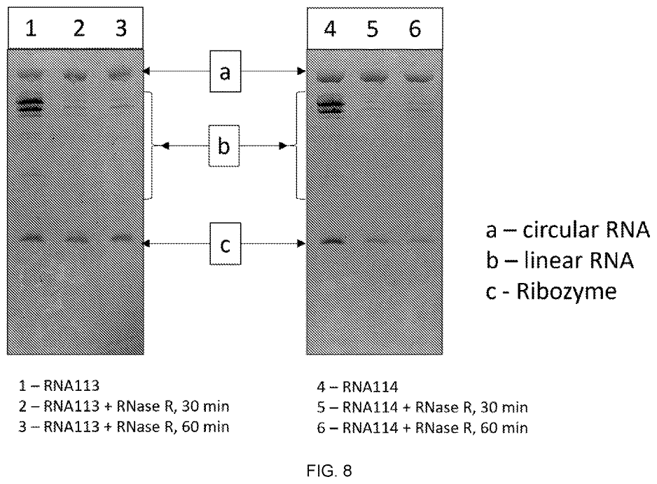

1 μg of RNA113 and RNA114 (generated from SBP113 and SBP114) is incubated with 1 U of Rnase R at 37° C. for different time intervals. A decrease in the intensity of linear RNA bands was observed within 30 min. However, the top band, concluded to be the circular RNA band, remains stable even after 60 min of incubation, as illustratively shown in FIG. 8.

As shown in FIG. 8, area “a” represents circular RNA, area “b” represents linear RNA, area “c” represents ribozyme, lane 1 corresponds to RNA113, lane 2 corresponds to RNA113 under 30 min treatment with Rnase R, lane 3 corresponds to RNA113 under 60 min treatment with Rnase R, lane 4 corresponds to RNA114, lane 5 corresponds to RNA114 under 30 min treatment with Rnase R, and lane 6 corresponds to RNA114 under 60 min treatment with Rnase R.

Linear RNA bands of both RNA113 and RNA114 are found to degrade within 30 min with circular RNA bands remaining even after 60 min of incubation with RNase R.

RNase R treatment is performed on purified IVT-generated RNA, without undergoing a circularization process. The analyses reveals that a sizable portion of IVT-generated RNA is circular.

### Example 6: Quantitation of Circular Vs Linear RNA

Quantitation of circularization is performed via an analysis of band intensity. The intensity of bands corresponding to circular RNA post-IVT and IVT+ circularization as seen on Urea-PAGE is indicative of circular RNA content. It is observed that few ribozyme variants (with a specific mutation or combination of mutations) perform better than others when compared to the wild-type ribozyme at generating circular RNA. RNA117, RNA119, and RNA120 respectively from SBP117, SBP119 and SBP120, show significantly higher amounts of circular RNA as compared to wildtype (RNA113 from SBP113).

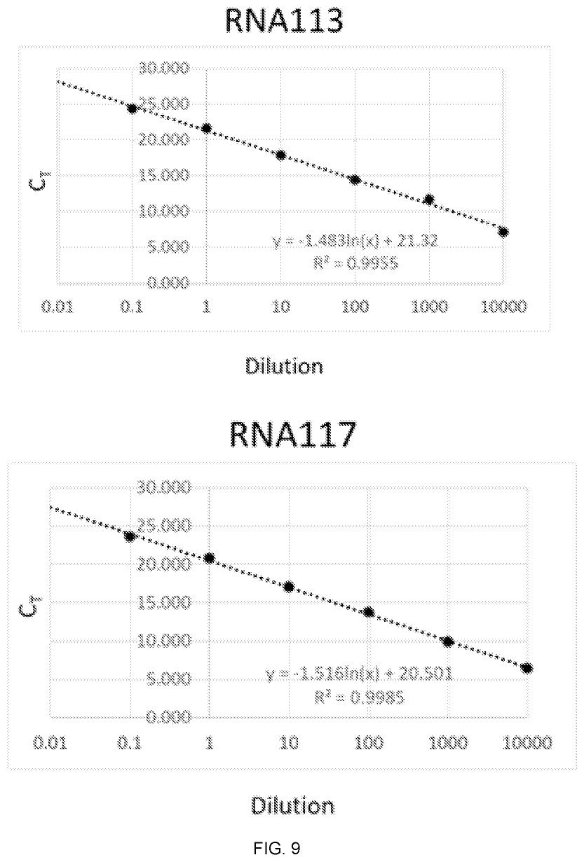

Quantification of circular RNA is alternatively performed using qRT-PCT. To quantify the amount of circular RNA present after an IVT reaction, qRT-PCR is performed. 1 μg of IVT RNA is used to generate cDNA using the manufacturer's protocol. Using convergent primer pair in the PCR reactions, a standard curve of Ct values against dilution of cDNA is obtained, as illustratively shown in FIG. 9. Ct value for cDNA using the divergent primer pair is used to determine the dilution of circular RNA in the RNA mixture from the standard curve. Using this approach, for every 1 μg of RNA, 0.1 μg of circular RNA113 and circular RNA114, and 0.25 μg of circular RNA117 are obtained.

### Example 7: Scarless Circularization

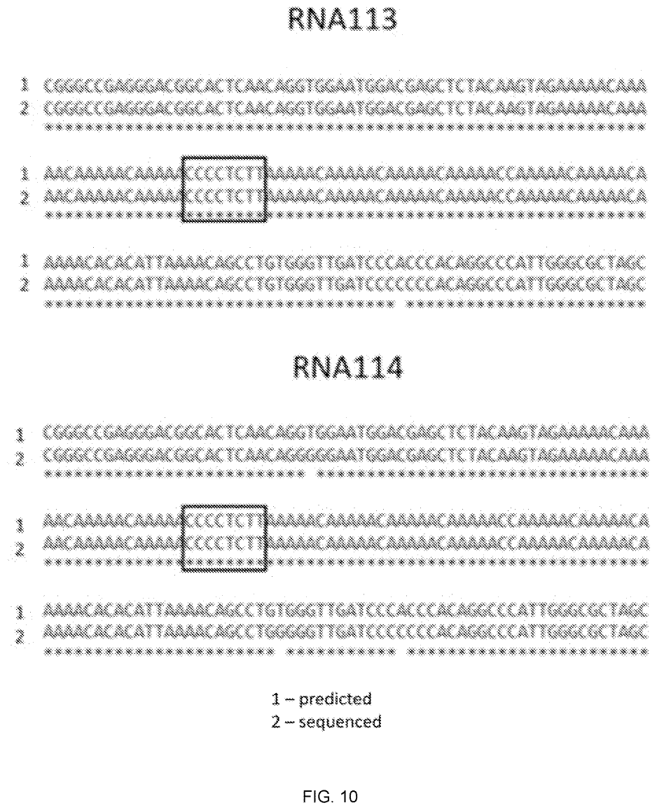

To assess if the circularization is without addition/deletion of nucleotides, the circularization junction is sequenced by Sanger sequencing using PCR products of the RNA from the divergent primer pair. For RNA113 and RNA114 the circularization junction match the designed sequences, as illustratively shown in FIG. 10. As shown in FIG. 10, line “1” represents predicted sequences, and line “2” represents sequenced sequences.

In contrast to the Ana PIE method, which often has a long circularization junction of ~40-60 nt, the method according to certain embodiment(s) of the present disclosure generates circular RNAs with a shorter nucleotide circularization junction, and in certain embodiment(s), the shorter nucleotide circulation junction is 5-8 nucleotides in length.

Additionally, in contrast to Ana PIE-generated circular RNA which contains sequences that are derived from Anabaena exons, the method according to certain embodiment(s) of the present disclosure generates circular RNA with no additional extraneous sequences, and hence carries minimal immunogenicity risk.

In certain embodiment(s), the IGS-Ribozyme-Gene of interest-target construct for IGS Sequence of interest is split such that the sequence downstream of the unique target sequence becomes 3′ end of the ribozyme.

In certain embodiment(s), the ribozyme sequence is of a length between 150 to 700, between 150 to 600, between 150 to 500, between 150 to 400, between 200 to 700, or between cover 244 to 387.

In certain particular embodiment(s), the nucleic acid construct includes a promoter sequence operably linked to other components of the construct, where the promoter sequence is recognized by an RNA polymerase enzyme for initiating transcription of the construct.

In certain particular embodiment(s), the nucleic acid construct includes an internal guide sequence (IGS) including two complementary sequences or structural motifs capable of base-pairing or interacting with each other, wherein the IGS facilitates the circularization of the transcribed RNA molecule by bringing the 5′ and 3′ ends of the RNA in close proximity.

In certain particular embodiment(s), the nucleic acid construct includes sequence encoding a group I intron ribozyme, where the ribozyme is a self-splicing RNA molecule capable of catalyzing its own excision from a larger RNA transcript.

In certain particular embodiment(s), the nucleic acid construct includes a sequence of interest encoding a desired RNA molecule, such as a coding RNA (for example, mRNA) or a non-coding RNA (for example, lncRNA, siRNA, miRNA), where the sequence of interest is split such that a portion downstream of the target sequence becomes the 3′ end of the ribozyme sequence.

In certain particular embodiment(s), the nucleic acid construct includes a target sequence complementary to the internal guide sequence, where the target sequence is located downstream of the sequence of interest and allows for base-pairing with the IGS, thereby bringing the 5′ and 3′ ends of the linear RNA molecule in close proximity for circularization.

In certain particular embodiment(s), the nucleic acid construct is designed to be transcribed in vitro to generate a linear RNA transcript. Upon incubation of the RNA transcript under appropriate conditions, the group I intron ribozyme undergoes self-splicing, facilitating the circularization of the RNA sequence without permutation of introns and exons, resulting in the formation of a scarless circular RNA molecule comprising the sequence of interest.

The present disclosure in certain embodiment(s) provides a versatile approach for producing scarless circular RNA molecules with desirable control over the sequence and structure of the circularized RNA. The circular RNA molecules thus generated may find implementation in various fields, including but not limited to gene expression studies, RNA therapeutics, and synthetic biology.

### Example 8: Mechanism for Generating Circular RNA

In certain embodiment(s), and as shown in Example 8, the designed RNA construct has the following elements in order from the 5′-end as shown in FIG. 1 and is employed for generating circular RNA.
Internal guide sequence (IGS)
Ribozyme sequence
Sequence of interest which includes an internal ribozyme entry site (IRES) and the gene of interest
Target sequence

Both the internal guide sequence (IGS) and target sequence are reverse complementary to each other and form the P1 loop of the Group I intron-based ribozyme. The base-paired region between the IGS and the target sequence also includes a G:U wobble base pairing, which is the site for the first nucleophilic attack, as shown in FIG. 3. The Group I intron-based ribozyme orchestrates a nucleophilic attack at the G:U wobble base pairing by the hydroxyl group of GTP, resulting in cleavage at the 3′ end of the target site (next to the U, shown at arrow 1). The free hydroxyl group thus formed at the 3′ end of the target site is directed by the ribozyme to perform a nucleophilic attack at its 3′ end (shown at arrow 2 in FIG. 3), thus resulting in the splicing out of the ribozyme and generation of a circular RNA with the sequence of interest.

FIG. 3 is a schematic diagram of the mechanism involved in the generation of circular RNA from an RNA construct containing the IGS, ribozyme, gene of interest, and target site region, where a schematic diagram of an RNA construct and a schematic diagram of a DNA construct are shown to include elements like the IGS, the ribozyme, the gene of interest, and the target site region.

### Example 9: Design of the DNA Construct

In certain embodiment(s), and as shown in Example 9 and further in view of FIG. 3, a T7 promoter sequence is included at the 5′ end of the DNA construct sequence to generate the RNA described in Example 1, by an in vitro transcription reaction. The DNA sequence additionally includes unique restriction sites not limited to EcoRI, SalI, and NdeI, at the 5′ and 3′ ends. Thus designed DNA sequence is synthesized and cloned into the pET9a (outsourced to GenScript) using the unique restriction sites to generate the circular RNA expression plasmid.

Multiple circular RNA expression plasmids are synthesized with ribozyme sequences derived from bacterial, fungal, or viral origin. Ribozymes that are experimentally validated to possess self-splicing ability are considered. Ribozyme sequences that are considered but not limited are listed below in Table 1.

TABLE 1
List of Ribozymes used in the circular RNA constructs

| SI No | Plasmid DNA | RNA | Ribosome origin | Subtype |
|---|---|---|---|---|
| 1 | SBP113 | RNA113 | Tetrahymena | IC-1 |
| | (SEQ. ID. | | | |
| | NO. 17) | | | |
| 2 | SBP124 | RNA124 | Anabaena | IC-3 |
| 3 | SBP125 | RNA125 | Azoarcus | IC-3 |
| 4 | SBP126 | RNA126 | Twort | IA-2 |
| | (SEQ. ID. | | | |
| | NO. 18) | | | |
| 5 | SBP127 | RNA127 | Scytalidium | IE-3 |
| 6 | SBP128 | RNA128 | Clostridium | IA-3 |

### Example 10: P1 Loop/Helix Formation

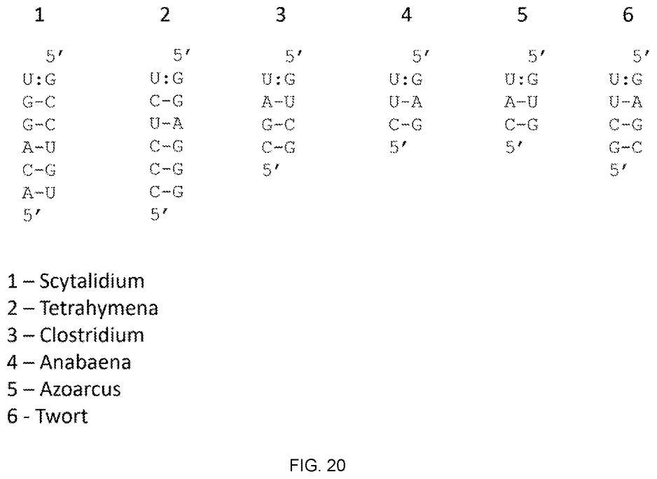

In certain embodiment(s), and as shown in Example 3 and further in view of FIG. 20, the RNA construct described in Example 10 generates circular RNA upon the formation of the P1 helix of the ribozyme. This helix contains a G:U wobble base-pairing which is essential for the splicing activity of the ribozyme in the presence of GTP. The length and the sequence of the P1 helix may vary based on the origin as well as the subtypes of the ribozyme sequence. As shown in FIG. 20, column “1” is directed to Scytalidium, column “2” is directed to Tetrahymena, column “3” is directed to Clostridium, column “4” is directed to Anabaena, column “5” is directed to Azoarcus, and column “6” is directed to Twort.

In certain embodiment(s), and as further shown in FIG. 20, the sequence of the P1 helix may vary without affecting the splicing activity of a particular ribozyme as long as
G:U wobble base-pairing is maintained
Length of the P1 loop base pairing is maintained.

### Example 11: In Vitro Transcription

In certain embodiment(s), and as shown in Example 11, the circular RNA expression plasmids are transformed into chemically competent Escherichia coli DH5alpha by heat shock method and propagated. The propagated plasmid is isolated using a plasmid purification midi kit (Qiagen) following the manufacturer's protocol.

Next, the isolated plasmid is linearized using the unique restriction enzyme site at the 3′ end of the DNA construct. The restriction enzymes that are used include but are not limited to SalI, NdeI, HindIII, and EcoRI. The linearization reaction is carried out at 37° C. for a minimum of 3 h (3 hours) and a maximum of 18 h. Linearization of plasmid DNA is confirmed by 0.8% agarose gel electrophoresis analyses and the linearized plasmid DNA is purified using a PCR purification kit (Qiagen) following the manufacturer's protocol. Subsequently, the concentration of the purified linearized plasmid DNA is determined using nanodrop (Thermo Fisher Scientific product) equipment.

The linearized circular RNA expression plasmids are transcribed in vitro using NEB's HiScribe T7 high-yield RNA synthesis kit (NEB) according to the manufacturer's protocol. The reaction is carried out at 37° C. at 20 uL scale (1 ug T7 DNA template, 1× Reaction buffer, 10 mM each ATP, UTP, CTP, GTP, T7 RNA polymerase mix 2 ul) for 3 h. Next, 1 uL of RNase-free DNase 1 (10 U/ul, Thermo Fisher Scientific product) is added and reacted at 37° C. for 30 minutes to remove the DNA template. Subsequently, the generated RNA is purified using a Monarch RNA cleanup kit (NEB) following the manufacturer's protocol and the concentration is measured using Nanodrop equipment.

1 μg each of the samples is mixed 1:1 with 2×RNA loading dye (NEB, 95% Formamide, 0.02% SDS, 0.02% bromophenol blue, 0.01% Xylene Cyanol, 1 mM EDTA), heated at 65° C. for 10 minutes, flash cooled on ice for 5 min, and then subjected to 4% polyacrylamide-8 M urea denaturing PAGE analyses (1 hours of electrophoresis at 250V). The gel is stained with a SYBR Safe DNA Stain product (Thermo Fisher Scientific) and analyzed using a Chemidoc (Biorad) instrument.

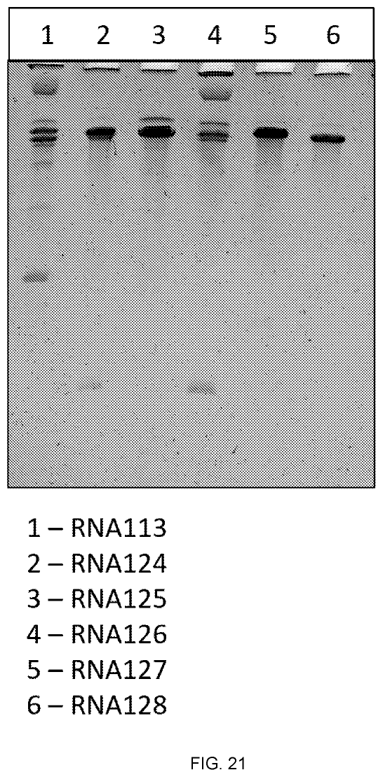

As observed in FIG. 21, RNA is generated from all the sampled circular RNA expression plasmids by the IVT reaction. In most samples, multiple bands corresponding to different sizes of the RNA are observed, with smaller RNA fragments found near the bottom of the gel and larger RNA fragments at the top of the gel. Only for two RNA samples, Lane 1 (Tetrahymena, marked “113”) and Lane 4 (Twort, marked “126”), a really large RNA fragment identified by the slowest migrating band at the top of the gel is observed. As shown in FIG. 21, lane “1” represents RNA113, lane “2” represents RNA124, lane “3” represents RNA125, lane “4” represents RNA126, and lane “5” RNA127, and lane “6” represents RNA128.

### Example 12: Determination of Circular RNA Presence—RNase R Treatment

In certain embodiment(s), and as shown in Example 5, to determine the presence of circular RNAs, the column-purified RNA samples are treated with RNase R. RNase R is an exo-ribonuclease that specifically cleaves linear RNA in the 3′ to 5′ direction.

RNase is an enzyme used in this example for the enrichment of circular RNA. Any other suitable agents or enzymes may be used for the enrichment of circular RNA.

1 uL of 10× RNase R reaction buffer (10× buffer: 0.2 M Tris-HCl pH 8.0, 1 M KCl, 1 mM MgCl2) and 1 Unit of RNase R are added to column-purified IVT (in vitro transcribed) RNA of up to 1 ug (adjusting the volume to 10 uL with water) and incubated at 37° C. and samples are taken at different timepoints.

Samples are mixed with 2× loading dye, and subjected to 4% polyacrylamide-8 M urea denaturing PAGE analyses.

After RNase R treatment it is observed that RNA generated from circular RNA expression plasmids containing ribozymes from Tetrahymena and Twort probably contain circular RNAs.

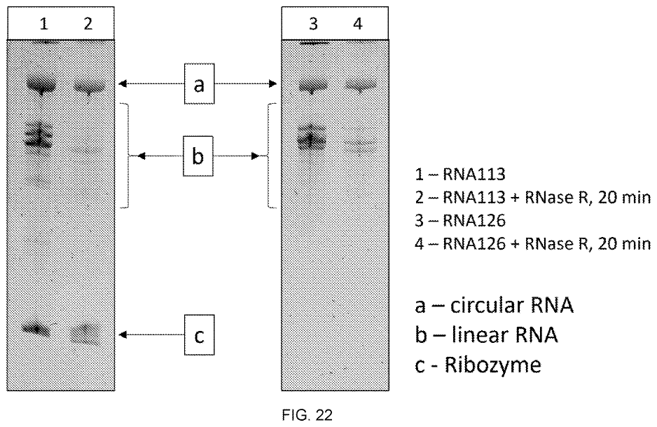

FIG. 22 is a schematic visualization of RNA samples from RNase R treatment after staining on 4% polyacrylamide-8M urea gel, where the slowest migrating band in both the samples are identified to correspond to circular RNA post-RNase R treatment, and where for both the constructs based on relative band intensity, about 30% circular RNA is observed before RNase R treatment, which is increased to above 80% after treatment. As shown in FIG. 22, area “a” represents circular RNA, area “b” represents linear RNA, area “c” represents ribozyme, lane “1” represents RNA113, lane “2” represents RNA113 under treatment with RNaseR for 20 minutes, lane “3” represents RNA126, and lane “4” represents RNA126 under treatment with RNase R for 20 minutes.

### Example 13: Determination of Circular RNA Presence—RT-PCR Approach

In certain embodiment(s), and as shown in Example 13, RT-PCR is performed to confirm that circular RNA was among the RNA bands not cleaved by RNase R from the PAGE results.

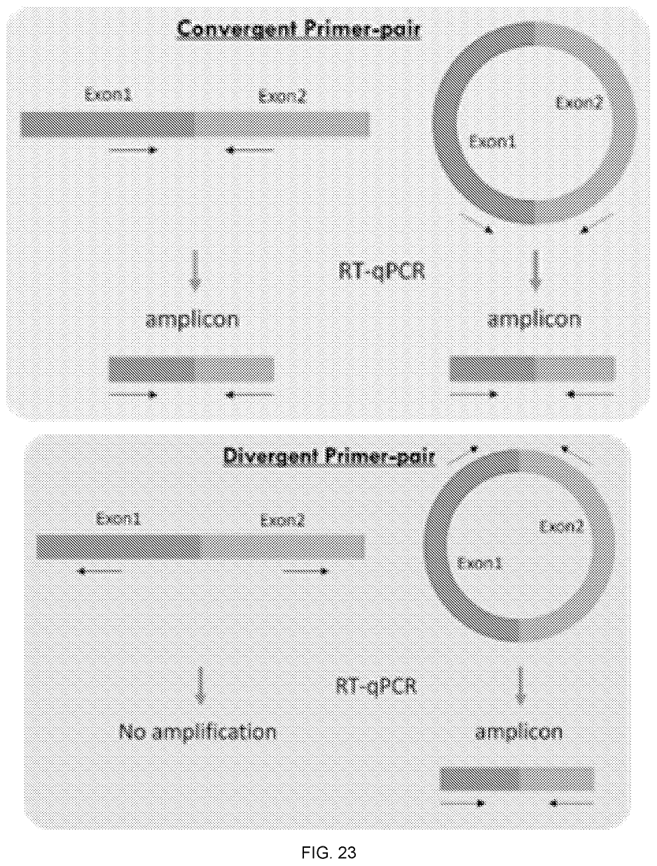

FIG. 23 is a schematic mechanism describing a procedure of RT-PCR using convergent and divergent primer pairs, where PCR product for a linear RNA is obtained when a convergent primer pair is used as compared to circular RNA, which can result in PCR product with both convergent and divergent primer pairs.

As illustratively shown in FIG. 23A, RT-PCR is performed using a
primer pair (convergent pair) capable of amplifying a PCR product both in linear and circular RNA form
primer pair (divergent pair) capable of PCR amplification only when circRNA (circular RNA) is present

RT-PCR is performed for column-purified IVT RNA from both RNA113 and RNA126. Before performing RT-PCR, cDNA for the column-purified IVT RNA samples is prepared using iScript™ cDNA Synthesis Kit (Biorad) following the manufacturer's protocol. Briefly, 1 μg of each RNA (RNA113 and RNA125) is added to 4 μl of 5×iSCript reaction mix, 1 μl of iScript Reverse Transcriptase. The samples are made up to a final volume of 20 μL, primed for 5 min at 20° C., and reacted at 46° C. for 20 minutes. Incubation at 95° C. for 1 minute inactivated the RT enzyme and the samples are stored at −20° C.

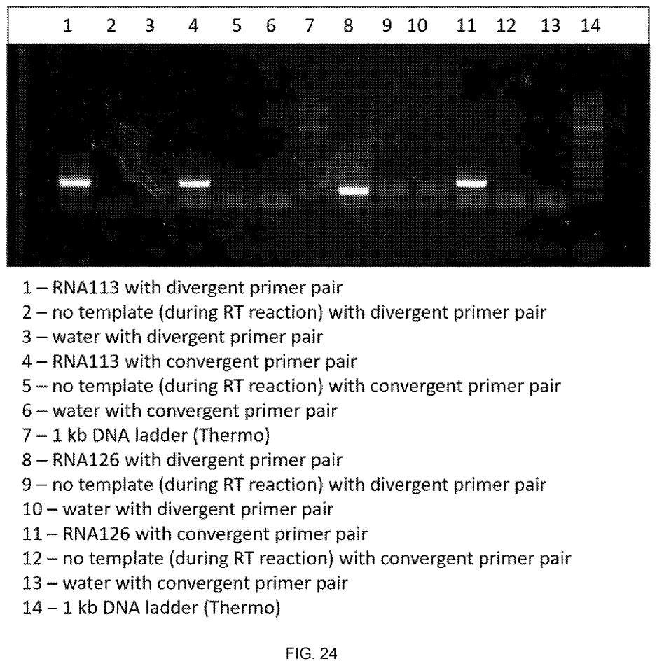

FIG. 24 is a schematic visualization of RT_PCR products obtained from convergent and divergent primer pairs on a 0.8% agarose gel, where PCR products at the expected size are observed, indicating circular RNA presence in both the samples. As shown in FIG. 24, lane “1” represents RNA113 with divergent primer set, lane “2” represents no template (during RT reaction) with divergent primer pair, lane “3” represents water with divergent primer pair, lane “4” represents RNA113 with convergent primer pair, lane “5” represents no template (during RT reaction) with convergent primer pair, lane “6” represents water with convergent primer pair, lane “7” represents 1 kb DNA ladder (Thermo), lane “8” represents RNA126 with divergent primer pair, lane “9” represents no template (during RT reaction) with divergent primer pair, lane “10” represents water with divergent primer pair, lane “11” represents RNA126 with convergent primer pair, lane “12” represents no template (during RT reaction) with convergent primer pair, lane “13” represents water with convergent primer pair, and lane “14” represents 1 kb DNA ladder (Thermo).

PCR is performed using EmeraldAmp® GT PCR Master Mix (Takara) using 1 uL of an RT sample. PCR is performed with both convergent primer pair and divergent primer pair, detailed in Table 2, at a concentration of 10 mM each.

TABLE 2
List of primers used

| SI | | |
|---|---|---|
| No | Name | Sequence (5′-3′) |
| 1 | Trans_circRNA_Div_F1 | AAGGACGGTGGACACTACGA |
| 2 | Trans_circRNA_Div_R1 | CTTTCTCCTTCAACCGCGTG |
| 3 | Trans_circRNA_Con_F1 | GGAAGGTTCTGTCAATGGGC |
| 4 | Trans_circRNA_Con_R1 | TCTTGGCCTTGTACGTCGTC |
| 5 | Trans_twort_cRNA_D_F1 | GACGACGTACAAGGCCAAGA |
| 6 | Trans_twort_cRNA_D_R1 | AAGTTACAGTTGGGGGAGGG |

PCR amplification is performed under the conditions of 95° C. for 1 minute, [95° C. for 30 seconds, 54° C. for 45 seconds, and 72° C. for 45 seconds], 35 cycles, 72° C. for 2 minutes. 10 uL of a PCR product is electrophoresed at 150 V for 35 minutes, and images are analyzed using a chemidoc. The expected lengths of the convergent and divergent primer pairs are listed below (Table 3).

TABLE 3
Sizes of the PCR products formed

| SI No | Name | Primer pair | Size (in bp) |
|---|---|---|---|
| 1 | Tetrahymena | Convergent | 500 |
| 2 | Tetrahymena | Divergent | 500 |
| 3 | Twort | Convergent | 500 |
| 4 | Twort | Divergent | 350 |

As observed from FIG. 6B, PCR products for both the RNA samples are observed with convergent and divergent primer pairs at the expected size, indicating that circular RNA are present in them.

### Example 14: Sequencing of Circularization Junction

In certain embodiment(s), and as shown in Example 14, the PCR products from the divergent primer pairs are purified according to the manufacturer's protocol using a PCR Purification kit (Qiagen). The purified PCR products are sequenced by Sanger sequencing (Service by BarCode Biosciences) using the primers listed in Table 2.

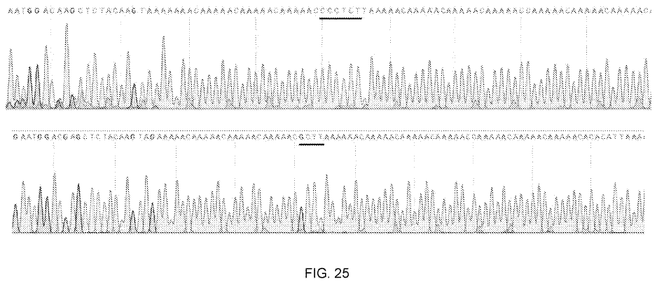

FIG. 25 is a schematic chromatogram from Sanger sequencing of PCR products, showing that the junction sequence matches what is designed for both Tetrahymena and Twort.

As illustratively shown in FIG. 25, the sequenced junctions match what is designed for both the ribozyme constructs.

### Example 15: IVT with Circular RNA Expressing Plasmids Carrying Mutations in the Ribozymes

In certain embodiment(s), and as shown in Example 15, the effect of mutations in the Tetrahymena ribozymes is assessed on the production of circular RNA. Table 4 lists the mutations that are assessed.

TABLE 4
plasmid-carrying mutations in the Tetrahymena ribozyme tested

| Plasmid | | |
|---|---|---|
| DNA | RNA | Description of ribozyme |
| SBP113 | RNA113 | Wild-type Tetrahymena ribozyme |
| SBP114 | RNA114 | P3 loop stabilized Tetrahymena ribozyme |
| SBP115 | RNA115 | P1extendedP10 loop forming Tetrahymena |
| | | ribozyme |
| SBP116 | RNA116 | Evolved Tetrahymena ribozyme |
| SBP117 | RNA117 | P1extendedP10 loop forming P3 loop |
| | | stabilized Tetrahymena ribozyme- |
| SBP118 | RNA118 | P1extendedP10 loop forming Evolved |
| | | Tetrahymena ribozyme |
| SBP119 | RNA119 | P3 loop stabilized + RNA 116 |
| SBP120 | RNA120 | P1extendedP10 loop forming P3 loop |
| | | stabilized evolved Tetrahymena ribozyme |

A DNA construct/template having the mutations was prepared. Plasmids capable of expressing each RNA construct is produced in the same manner as in Example 1, and in vitro transcription reactions are performed at 37° C. for 3 hours in the same manner as described in Example 11.

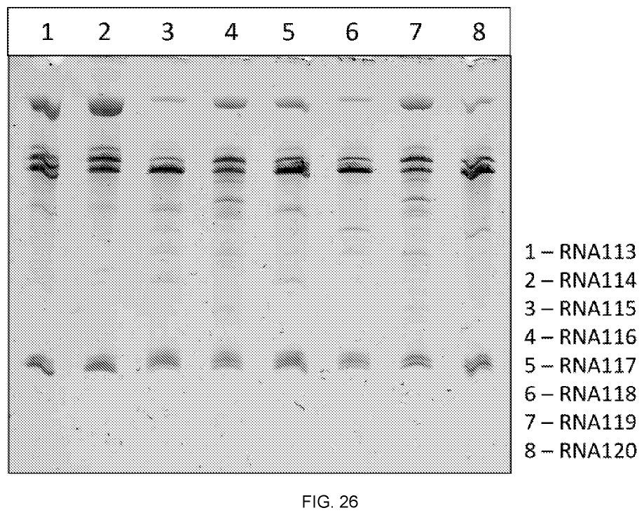

The amount of circular RNA formed is compared and identified by relative band intensity by performing PAGE on 4% polyacrylamide-8 M urea gel. As illustratively shown in FIG. 26, all the designed ribozymes generate circular RNA, however, the amount of circular RNA made vary across the constructs. As shown in FIG. 26, lane “1” represents RNA113, lane “2” represents RNA114, lane “3” represents RNA115, lane “4” represents RNA116, lane “5” represents RNA117, lane “6” represents RNA118, lane “7” represents RNA119, and lane “8” represents RNA120.

FIG. 26 is a schematic visualization of RNA after staining on a 4% polyacrylamide-8M urea gel, where RNA from all the sampled circular RNA expression plasmids with mutation carrying Tetrahymena ribozyme is generated by IVT (in vitro transcription).

The amount of circular RNA made or circularization efficiency is determined by the ratio of the band intensity of the circular RNA band to the band intensity of all RNA bands in a particular lane of the polyacrylamide-8 M urea gel. Anywhere between 6% to 32% of circular RNA is formed directly after IVT reactions in these constructs. Additional circularization steps do not increase the yield of circular RNA.

### Example 16: RT-PCR to Determine the Presence of Circular RNA in RNA Samples Generated Using Mutation-Carrying Ribozymes

RT-PCR for all the sampled constructs is performed to determine the presence of circular RNA as described in Example 13. PCR is performed with convergent and divergent primer pairs for Tetrahymena mentioned in Table 2. PCR products at the expected size are observed for all the designed constructs with both convergent and divergent primer pairs, indicating the presence of circular RNA. Results are shown in FIG. 7A and FIG. 7B.

### Example 17: RNase R Treatment to Determine the Presence of Circular RNA in RNA Samples Generated Using Mutation-Carrying Ribozymes

In certain embodiment(s), and as shown in Example 17, to confirm the presence of circular RNAs in the column-purified RNA samples, RNase R treatment is performed according to methods described in Example 12 and in Example 15. In particular, 1 uL of 10×RNase R reaction buffer (10× buffer: 0.2 M Tris-HCl pH 8.0, 1 M KCl, 1 mM MgCl2) and 1 Unit of RNase R are added to column-purified IVT RNA of up to 1 ug (adjusting the volume to 10 uL with water) and incubated at 37° C. for 20 min.

Samples are mixed with 2× loading dye, and subjected to 4% polyacrylamide-8 M urea denaturing PAGE analyses.

After RNase R treatment it is observed that RNA generated from a few of the circular RNA expression plasmids carrying mutation-containing ribozymes contained circular RNAs.

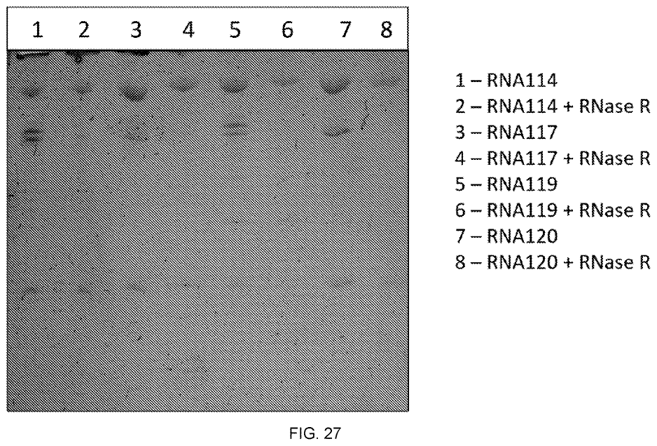

Further in view of FIG. 27, the large RNA fragment identified by the slowest migrating band at the top of the gel does not get degraded in the presence of RNase R and demonstrates a correspondence to circular RNA in both samples, in contrast to other bands in the lane. As shown in FIG. 27, lane “1” refers to RNA114, lane “2” refers to RNA114 under treatment with RNase R, lane “3” refers to RNA117, lane “4” refers to RNA117 under treatment with RNase R, lane “5” refers to RNA119, lane “6” refers to RNA119 under treatment with RNase R, lane “7” refers to RNA120, and lane “8” refers to RNA120 under treatment with RNase R.

FIG. 27 is a schematic visualization of RNA samples from RNase R treatment after staining on 4% polyacrylamide-8M urea gel, where the slowest migrating band in both the samples is identified to correspond to circular RNA post-RNase R treatment, and where for all of these constructs based on relative band intensity, anywhere between 6% to 32% circular RNA is observed before RNase R treatment, which is increased to >80% after the RNase R treatment.

### Example 18: Mechanism for Generating Scarless Circular RNA

In certain embodiment(s), a circular RNA is generated, including a circularization junction sequence of 4 to 6 nucleotides (scar sequence) in addition to the sequence of interest.

In certain embodiment(s), generated is a circular RNA consisting only of the IGS, the ribozyme, the sequence of interest (GOI), and uracil base at the 3′ end.

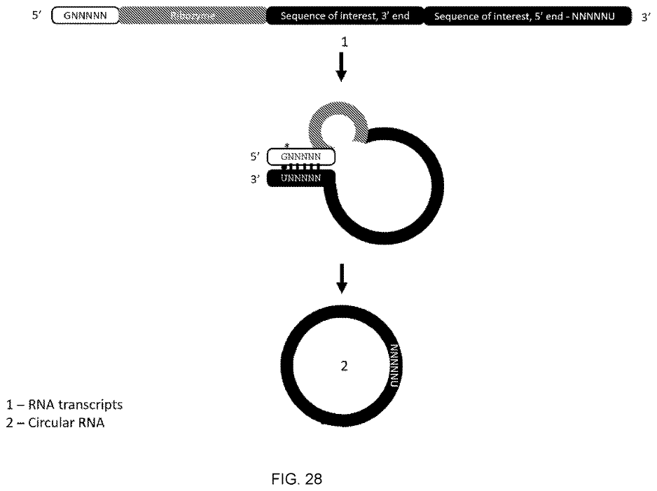

FIG. 28 is a schematic diagram of the RNA construct and the generated circular RNA. As shown in FIG. 28, numeral “1” refers to RNA transcripts, and numeral “2” refers to circular RNA.

According FIG. 28, in the sequence of interest when uracil is present after 3 to 5 unique nucleotide sequences, that sequence is be part of the 5′ end of the RNA construct design. The remaining sequence of interest at the 3′ end of the uracil is included behind the ribozyme in the RNA construct design. Using this design, circular RNA is generated with only the sequence of interest without addition of scars.

### Example 19: IVT of the Scarless Circular RNA Expression Plasmids

For the RNA constructs described in Example 18, DNA constructs according to Table 5 are designed as per Example 9 and synthesized. In vitro transcription reactions for these plasmids are performed at 37° C. for 3 hours in the same manner as described in Example 11.

The amount of circular RNA formed is compared and identified by relative band intensity by performing PAGE on 4% polyacrylamide-8 M urea gel.

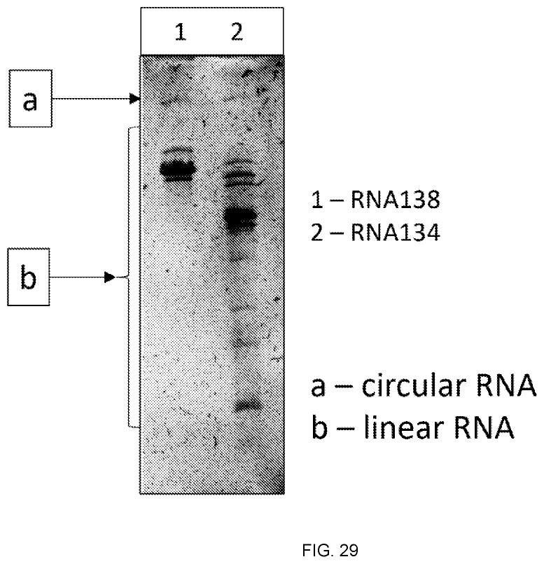

As illustratively shown in FIG. 29, all the designed ribozymes generate circular RNA; however, the amount of circular RNA thus made vary across the constructs. The amount of circular RNA formed by these constructs is lower than that formed by the constructs referenced in Examples 11 and 15. This observation indicates to the fact that the location of the uracil plays an important role in determining the amount of circular RNA that is formed after the IVT reaction. As shown in FIG. 29, area “a” refers to circular RNA, area “b” refers to linear RNA, numeral “1” refers to RNA138, and numeral “2” refers to RNA134.

FIG. 29 is a schematic visualization of RNA after staining on a 4% polyacrylamide-8M urea gel, where RNA from the designed scarless circular RNA expression plasmids is generated by IVT, showing relatively lower amounts of circular RNA formation observed in these constructs after IVT reaction.

TABLE 5
List of scarless circular RNA expressing plasmid that are tested
including the ribozyme origin as well as the target sequence.

| | | Ribozyme | | |
|---|---|---|---|---|
| | | sequence | | Target |
| DNA | RNA | derived from | Description | sequence |
| SBP134 | RNA134 | RNA113 | Tetrahymena | 5′-CCCUCU-3′ |
| | | | scarless construct | |
| SBP138 | RNA138 | RNA126 | Twort scarless | 5′-GCUU-3′ |
| | | | construct | |

In certain embodiment(s), one or more of SBP113, SBP124, SBP125, SBP126, SBP1127, and SBP128 contain ribozyme sequences that are self splicing.

In certain embodiment(s), ribozymes in SBP113 and SBP126 are active and capable for self-circularization.

In certain embodiment(s), SBP114, SBP115, SBP116, SBP117, SBP118, SBP119, SBP120, and SBP121 are each a variant (with one or more mutations in the sequence) of ribozyme in SBP113. Some of these variants may be able to self-splicing but not for self-circularization.

In certain embodiment(s), some constructs like SBP114, are as active if not better at generating circular RNA as compared to SBP113.

In certain embodiment(s), SBP134 and SBP138 contain the same ribozymes found in SBP113 and SBP126, respectively and generate circular RNA that are completely scar-less.

Although the present disclosure has been shown and described with reference to exemplary embodiments, those skilled in the art understand that, without departing from the spirit and scope of the present disclosure as defined by the appended claims and their equivalents, various modifications in form and detail may be made to the present disclosure. Therefore, the scope of the present disclosure should not be limited to the embodiments described herein, but should be determined not only by the appended claims, but also by the equivalents of the appended claims.

What is claimed is:

1. A method of making circularization of an RNA of interest without a use of permuted intron-exon (PIE), the method comprising:
providing a DNA (deoxyribonucleic acid) construct, wherein the DNA construct includes a Group I intron-based ribozyme, a sequence of interest, and a target sequence;
causing an RNA to be transcribed from the DNA construct, the RNA includes a segment S1 corresponding to the ribozyme, a segment S2 corresponding to the target sequence, and a segment S3 corresponding to the sequence of interest; and
effecting a base-pairing between the segment S1 and the segment S2, wherein an occurrence of the base-pairing induces circularization of the segment S3.

2. The method of claim 1, wherein the circularization of the segment S3 comprises:
moving segment S1 to a 5′ end of the segment S3 and keeping the segment S2 at a 3′ end of the RNA, to generate a scarless circular RNA.

3. The method of claim 1, wherein the base-pairing between the segment S1 and segment S2 includes a wobble base pairing G:U.

4. The method of claim 1, wherein the Group I intron-based ribozyme is a Tetrahymena ribozyme or a Twort ribozyme.

5. The method of claim 1, wherein the Group I intron-based ribozyme is a variant of Tetrahymena.

6. The method of claim 1, wherein the segment S1 or the segment S2 is GNNNNN or NNNNNU, G is guanine, U is uracil, N is any one of adenine, guanine, cytosine, and uracil.

7. The method of claim 1, wherein the segment S1 or the segment S2 is GGAGGG or CCCUCU, G is guanine, U is uracil, A is adenine, and C is cytosine.

8. The method of claim 1, wherein the Group I intron-based ribozyme is Twort, and the segment S1 or the segment S2 is GNNN or NNNU, G is guanine, U is uracil, N is any one of adenine, guanine, cytosine, and uracil.

9. The method of claim 1, wherein the Group I intron-based ribozyme is Twort, and the segment S1 or the segment S2 is GAGC or GCUU, G is guanine, U is uracil, A is adenine, and C is cytosine.

10. The method of claim 1, further comprising:
detecting a circular RNA of the RNA as transcribed in a PCR (polymerase chain reaction) using both a pair of divergent primers and a pair of convergent primers, wherein the pair of convergent primers are oriented in opposite directions (5′ to 3′ and 3′ to 5″) and allow an amplification to proceed inwardly towards each other, wherein the pair of divergent primers are oriented in a same direction (both from 5′ to 3′ or both 3′ to 5′) and allow an amplification to proceed outwardly away from each other.

11. An isolated nucleic acid, comprising: a nucleic acid sequence with at least 80% (80 percent) similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21 wherein the at least 80% similarity refers to that at least 8 nucleotides are the same for every 10 nucleotides between two sequences, in a 5′ to 3′ direction.

12. The isolated nucleic acid of claim 11, wherein the nucleic acid sequence is with at least 90% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21 wherein the at least 90% similarity refers to that at least 9 nucleotides are the same for every 10 nucleotides between two sequences, in a 5′ to 3′ direction.

13. The isolated nucleic acid of claim 11, wherein the nucleic acid sequence is SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21.

14. The isolated nucleic acid of claim 11, wherein the nucleic acid sequence is with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, or SEQ ID NO. 20.

15. The isolated nucleic acid of claim 11, wherein the nucleic acid sequence is with at least 80% similarity to SEQ ID NO. 12 or SEQ ID NO. 21.

16. A DNA (deoxyribonucleic acid) construct, comprising: a ribozyme sequence, wherein an RNA segment transcribed from the ribozyme sequence includes a nucleic acid sequence with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21, wherein the at least 80% similarity refers to that at least 8 nucleotides are the same for every 10 nucleotides between two sequences, in a 5′ to 3′ direction.

17. The DNA construct of claim 16, wherein the RNA segment transcribed from the ribozyme sequence is segment S1, and the DNA construct further comprises:
a target sequence, wherein an RNA segment transcribed from the target sequence is segment S2, and wherein segment S1 is complementary in base pair to the segment S2.

18. The DNA construct of claim 17, wherein a wobble base pair G-U is present between the segment S1 and the segment S2.

19. The DNA construct of claim 17, further comprising:
a sequence of interest, positioned between the ribozyme sequence and the target sequence.

20. A method of forming circular RNA (ribonucleic acid), the method comprising:
providing a DNA (deoxyribonucleic acid) construct including a ribozyme sequence, wherein an RNA segment transcribed from the ribozyme sequence includes a nucleic acid sequence with at least 80% similarity to SEQ ID NO. 01, SEQ ID NO. 02, SEQ ID NO. 03, SEQ ID NO. 04, SEQ ID NO. 05, SEQ ID NO. 06, SEQ ID NO. 07, SEQ ID NO. 08, SEQ ID NO. 09, SEQ ID NO. 12, SEQ ID NO. 20, or SEQ ID NO. 21 wherein the at least 80% similarity refers to that at least 8 nucleotides are the same for every 10 nucleotides between two sequences, in a 5′ to 3′ direction.


SEQUENCE LISTING (23 sequences)

SEQ ID NO: 1 - RNA, 393 nt, synthetic construct
  source 1..393: mol_type = other RNA
ggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 2 - RNA, 393 nt, synthetic construct
  source 1..393: mol_type = other RNA
ggagggaaaagttatcaggcatgcacctggtagctagtctttaaaccaatagattgcatcggtttaaaaggcaagaccgccaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatggcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 3 - RNA, 402 nt, synthetic construct
  source 1..402: mol_type = other RNA
ggtacctttggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 4 - RNA, 398 nt, synthetic construct
  source 1..398: mol_type = other RNA
ggtctggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatctgtatgcgaaagtatattgattagttttggagtactcg

SEQ ID NO: 5 - RNA, 402 nt, synthetic construct
  source 1..402: mol_type = other RNA
ggtacctttggagggaaaagttatcaggcatgcacctggtagctagtctttaaaccaatagattgcatcggtttaaaaggcaagaccgccaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatggcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 6 - RNA, 402 nt, synthetic construct
  source 1..402: mol_type = other RNA
ggtacctctggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatctgtatgcgaaagtatattgattagttttggagtactcg

SEQ ID NO: 7 - RNA, 398 nt, synthetic construct
  source 1..398: mol_type = other RNA
ggtctggagggaaaagttatcaggcatgcacctggtagctagtctttaaaccaatagattgcatcggtttaaaaggcaagaccgccaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatggcggtcggggaagatgtattcttctcataagatatagtcggacctctccttaatgggagctagcggatgaagtgatgcaacactggagccgctgggaactaatctgtatgcgaaagtatattgattagttttggagtactcg

SEQ ID NO: 8 - RNA, 402 nt, synthetic construct
  source 1..402: mol_type = other RNA
ggtacctctggagggaaaagttatcaggcatgcacctggtagctagtctttaaaccaatagattgcatcggtttaaaaggcaagaccgccaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatggcggtcggggaagatgtattcttctcataagatatagtcggacctctccttaatgggagctagcggatgaagtgatgcaacactggagccgctgggaactaatctgtatgcgaaagtatattgattagttttggagtactcg

SEQ ID NO: 9 - RNA, 324 nt, synthetic construct
  source 1..324: mol_type = other RNA
ggagggaaaagttatcaggcatgcacctggtagctagtctttaaaccaatagattgcatcggtttaaaaggcaagaccgtcaaattgcgggaaaggggtcaacagacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 10 - RNA, 241 nt, synthetic construct
  source 1..241: mol_type = other RNA
gagccttagagaagaaattctttaagtggatgctctcaaactcagggaaacctaaatctagctatagacaaggcaatcctgagccaagccgaagtagtaattagtaagttaacaacagataacttacagctagtcggaaggtgcagagactcgacgggagctaccctaacgtcaagacgagggtaaagagagagtccaattctcaaagccaataggcagtagcgaaagctgcgggagaatg

SEQ ID NO: 11 - RNA, 197 nt, synthetic construct
  source 1..197: mol_type = other RNA
gtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaaacgctatggtgaaggcatagtccagggagtggcgaaagtcacacaaaccgg

SEQ ID NO: 12 - RNA, 244 nt, synthetic construct
  source 1..244: mol_type = other RNA
gagcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctatccgtaacataattg

SEQ ID NO: 13 - RNA, 382 nt, synthetic construct
  source 1..382: mol_type = other RNA
gcctgtgacagcggggcttcgaacttttccgtgttttttttttttcttccgctagtcgatcatccgtgacggccgggcaagcgcccgagtaccaggaccgtcgggcccccgagaagggggggcctaggatgcggcaagacgacccggttcggggaacgccagcgtgcgctggccgatcccgaggcgaggtgccgtagcgggcacccgtcgtaacgcgcggtagggcgtcggtcccccctcccacggcggaggggaggcttaaggtacgtgctaaacccccagcgatggggcctgtaggaaaagccctggtacggcgaagcctacggggaccgcccgatggcggtcggatgccggcgggccacggagcccccggcgtcgattg

SEQ ID NO: 14 - RNA, 279 nt, synthetic construct
  source 1..279: mol_type = other RNA
gtcgccttatgaagtgattactaagtgaaaacttggctttatcggtgaaaccgaaacgtaaagacgtcggcaataccgagtggtatgtggggaaacccaacagccgtgtatcgactcataggtttctaaccttaagaaatgaaattttcttaagtaagaaatactaggatagaagttttaaactataatatagcttctataacatgccaaggtacttaaaacttataagttttaagcacgagatatagtcagtgccattagagataatggaatagcatg

SEQ ID NO: 15 - DNA, 19 bp, synthetic construct
  source 1..19: mol_type = other DNA
taatacgactcactatagg

SEQ ID NO: 16 - DNA, 711 bp, synthetic construct
  source 1..711: mol_type = other DNA
atggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcgggcctcttccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttacaatgttaatatcaagcttgacatcacttcccacaatgaggactacacaatcgttgagcaatacgagcgggccgagggacggcactcaacaggtggaatggacgagctctacaagtag

SEQ ID NO: 17 - DNA, 1959 bp, synthetic construct
  source 1..1959: mol_type = other DNA
gaattccagctgtaatacgactcactataggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgtaaaaacaaaaacaaaaacaaaaaccaaaaacaaaaacaaaaacacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcgggcctcttccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttacaatgttaatatcaagcttgacatcacttcccacaatgaggactacacaatcgttgagcaatacgagcgggccgagggacggcactcaacaggtggaatggacgagctctacaagtagaaaaacaaaaacaaaaacaaaaacccctctgtcgac

SEQ ID NO: 18 - DNA, 1814 bp, synthetic construct
  source 1..1814: mol_type = other DNA
gaattccagctgtaatacgactcactataggaagagcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctatccgtaacataattgaaaaaacaaaaacaaaaacaaaaaccaaaaacaaaaacaaaaacacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcgggcctcttccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttacaatgttaatatcaagcttgacatcacttcccacaatgaggactacacaatcgttgagcaatacgagcgggccgagggacggcactcaacaggtggaatggacgagctctacaagtagaaaaacaaaaacaaaaacaaaaacgcttttgtcgac

SEQ ID NO: 19 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 20 - RNA, 1922 nt, synthetic construct
  source 1..1922: mol_type = other RNA
ggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgtccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttacaatgttaatatcaagcttgacatcacttcccacaatgaggactacacaatcgttgagcaatacgagcgggccgagggacggcactcaacaggtggaatggacgagctctacaagtagaaaaacaaaaacaaaaacaaaaacaaaaacaaaaacaaaaacaaaaaccaaaaacaaaaacaaaaacacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcggccctctgtcga

SEQ ID NO: 21 - RNA, 1780 nt, synthetic construct
  source 1..1780: mol_type = other RNA
ggaagagcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctatccgtaacataattgacaatgttaatatcaagcttgacatcacttcccacaatgaggactacacaatcgttgagcaatacgagcgggccgagggacggcactcaacaggtggaatggacgagctctacaagtagaaaaacaaaaacaaaaacaaaaacaaaaaacaaaaacaaaaacaaaaaccaaaaacaaaaacaaaaacacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcgggcctcttccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttttgtcga

SEQ ID NO: 22 - DNA, 1952 bp, synthetic construct
  source 1..1952: mol_type = other DNA
gaattccagctgtaatacgactcactataggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcgtccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttacaatgttaatatcaagcttgacatcacttcccacaatgaggactacacaatcgttgagcaatacgagcgggccgagggacggcactcaacaggtggaatggacgagctctacaagtagaaaaacaaaaacaaaaacaaaaacaaaaacaaaaacaaaaacaaaaaccaaaaacaaaaacaaaaacacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcggccctctgtcgac

SEQ ID NO: 23 - DNA, 1810 bp, synthetic construct
  source 1..1810: mol_type = other DNA
gaattccagctgtaatacgactcactataggaagagcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctatccgtaacataattgacaatgttaatatcaagcttgacatcacttcccacaatgaggactacacaatcgttgagcaatacgagcgggccgagggacggcactcaacaggtggaatggacgagctctacaagtagaaaaacaaaaacaaaaacaaaaacaaaaaacaaaaacaaaaacaaaaaccaaaaacaaaaacaaaaacacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtcagcaagggcgaggaagacaatatggcaatcatcaaggagttcatgagattcaaggttcacatggaaggttctgtcaatgggcacgagttcgagatcgagggagagggagagggaaggccatacgagggaacacaaacagccaagttaaaggtcacgaagggcgggcctcttccattcgcatgggacatattaagtcctcaattcatgtacgggtctaaggcatacgttaagcaccccgcagacataccagactacttgaagctctccttccccgagggattcaagtgggagagggtcatgaatttcgaggacggtggggtagttacggttactcaagactcttccttacaagacggagagttcatatacaaggttaagttacggggcactaatttccctagtgacgggccagttatgcaaaagaagacgatgggatgggaagccagttcagagaggatgtacccagaggacggtgccttaaagggagagatcaagcaacgactcaagttgaaggacggtggacactacgacgctgaggtaaagacgacgtacaaggccaagaagccagtacaactccccggagcttttgtcgac